(19) United States
(12) Patent Application Publication
(10) Pub. No.: US 2023/0174909 A1
(43) Pub. Date: Jun. 8, 2023

(54) CELL CULTURE SYSTEM FOR PERFUSABLE NETWORKS OF SELF-ASSEMBLED CELLS

(71) Applicant: MCMASTER UNIVERSITY, Hamilton, (CA)

(72) Inventors: Boyang ZHANG, Oakville (CA); Shravanthi RAJASEKAR, Ancaster (CA); Dawn song YI LIN, Scarborough (CA)

(21) Appl. No.: 17/997,991

(22) PCT Filed: May 6, 2021
(86) PCT No.: PCT/CA2021/050636

§ 371 Date: Nov. 4, 2022

Related U.S. Application Data
(60) Provisional application No. 63/020,805, filed on May 6, 2020.

(51) Int. Cl. C12M 1/32; C12M 1/00; C12M 1/12

(52) U.S. Cl. CPC C12M 23/12; C12M 29/10; C12M 25/14

(57) ABSTRACT

Described herein is a cell culture system for constructing a perfusable network of self-assembled cells comprising a multi-well plate embedded with microchannels connecting a central well with at least one inlet well and at least one outlet well, the central well for culturing seeded cells within an extracellular matrix, wherein the perfusable network allows perfusion through the microchannels connecting the central well with at least one inlet well and at least one outlet well. The cell culture system allows the array of perfusable networks formed, connected, and perfused inside the multi-well plate to be accessible and/or extractable from the top of the central well. In aspect, the cell culture system can improve the experimental throughputs of organ-on-a-chip systems and expand the application of microphysiological systems to regenerative cell therapy. A perfusable network of self-assembled cells and method of making thereof using the cell culture system described herein are also provided.

### FIELD

The present disclosure relates to tissue engineering, and in particular, a cell culture system to culture, perfuse and assay perfusable networks of self-assembled cells.

### BACKGROUND

Blood vessels play vital roles in organ function and development. Vascular systems not only supply tissues with oxygen and nutrients, but also participate in many biological processes, such as transporting immune cells in inflammatory response, trafficking cancer cells in tumor metastasis, establishing biochemical gradients, improving parenchymal tissue survival and function through paracrine signaling, and repairing tissues through angiogenesis, etc. The incorporation of vascular networks in biological models is integral to accurately model human diseases as well as to maintain proper tissue function in vitro. Microphysiological systems, also known as organ-on-a-chip, have been developed to model various cellular microenvironments in the human body¹. For instance, Huh et al. developed a lung alveoli-on-a-chip, lung airway-on-a-chip, and placenta-on-a-chip, etc²⁻⁴. These organ chips consist of multi-layer microfluidic channels separated by a porous membrane that are lined with epithelium cells and human endothelial cells to model the epithelial and vascular interface of various organs. The membrane based vascular barrier is advantageous for tracking drug and biomolecular transports, immune cell extravasation, as well as simulating blood circulation.

Microfluidic systems have also been developed to culture microvascular networks to study angiogenesis, vasculogenesis and various vascular events by taking advantage of the ability of endothelial cells to self-assemble into a microvascular network given the right conditions⁵. This type of system allows for the intimate contact between the endothelial cells and other stromal and parenchymal cells and is particularly useful for studying the remodeling process of blood vessels in details in response to the chemical gradient, fluid flow, and drug treatments^{6,7} However, these self-assembled vascular networks are often constrained inside a closed or semi-closed microfluidic channel that limits their physical integration upon implantation or with other larger tissues models such as organoids^{8,9}. Organoids are a 3D cell culture of self-organized differentiating cells that can recapitulate in vivo morphology and cell organization on a smaller scale, while also displaying genetic fingerprints very similar to that of the original tissue₁₀, which are usually grown on natural extracellular matrix (ECM) to provide the structural support for cell attachment and organization¹¹.

While the main advantage of an organoid is structural sophistication, this approach is limited by the lack of vascularization and perfusion^{12,13}. Organoids are usually cultured inside static multi-well plates. Even though vasculature is prevalent in the body, many organoid model systems completely omit the vascular network and in those that do contain vasculature, the vascular networks are usually non-perfusable or not intra-vascular perfused. Important biological events, such as intercellular crosstalk, immune cell migration, and biomolecular transport, occur at the tissue vascular and epithelium interface. Without a way to access this biological interface and/or the intra-vascular space, the organoid models cannot be fully functional. Similarly, accelerating vascular connection upon tissue implantation is critically important to ensuring tissue survival in regenerative medicine¹³. Previously, patterned vasculature has been shown to improve vascular integration in vivo but has not been pre-perfused in vitro prior to implantation¹⁴. Other systems that allow the culture of perfusable microvasculature in vitro do not allow for easy tissue extraction for implantation⁸. In the case of both drug testing and therapeutic implantation, a perfusable cell culture device containing self-assembled vasculature networks is needed to address the required demands.

### SUMMARY

In accordance with an aspect, there is provided an open chamber for cell culture, the chamber comprising an inlet and an outlet for non-tangential flow of fluid from the inlet to the outlet.

In an aspect, the chamber is an open cylinder.

In an aspect, the chamber comprises an open top and a closed bottom and wherein the open top and closed bottom have substantially equal diameters.

In an aspect, the chamber does not comprise a sacrificial material.

In an aspect, the chamber does not comprise a membrane.

In an aspect, the inlet and/or outlet is a channel.

In an aspect, the channel is about 0.5 to about 3 times as wide as it is tall.

In an aspect, the channel is about 200 microns wide and about 100 to about 200 microns tall.

In an aspect, the inlet leads to an inlet chamber and/or the outlet leads to an outlet chamber.

In an aspect, the inlet chamber and/or the outlet chamber are open or closed.

In an aspect, the open chamber comprises at least two inlets and/or at least two outlets.

In an aspect, the open chamber comprises a patterned base.

In an aspect, the patterned base comprises connected grooves for guiding self-assembly of cultured cells.

In an aspect, the open chamber is configured for unidirectional or bidirectional fluid flow from the inlet to the outlet.

In an aspect, the open chamber further comprises a hydrogel on a bottom surface of the open chamber.

In an aspect, the hydrogel comprises a fibrin matrix, fibrin, Matrigel, collagen I, a decellularized matrix, or a combination thereof.

In an aspect, the open chamber further comprises cells seeded into the open chamber.

In accordance with an aspect, there is provided an array comprising the open chamber described herein and at least one inlet chamber and at least one outlet chamber.

In accordance with an aspect, there is provided a multi-well plate comprising the array described herein.

In an aspect, the multi-well plate comprises a plurality of the arrays described herein.

In accordance with an aspect, there is provided a method for making a perfusable network of self-assembled cells, the method comprising applying a hydrogel and cells to the open chamber described herein and culturing the cells.

In an aspect, the hydrogel and the cells are applied together as a mixture.

In an aspect, the hydrogel and the cells are applied sequentially.

In an aspect, the method further comprises flowing fluid from the inlet to the outlet.

In accordance with an aspect, there is provided a heterogenous perfusable network of self-assembled cells.

In accordance with an aspect, there is provided a cell culture system for constructing a perfusable network of self-assembled cells comprising a multi-well plate embedded with microchannels connecting a central well with at least one inlet well and at least one outlet well, the central well for culturing seeded cells within an extracellular matrix, wherein the perfusable network allows perfusion through the microchannels connecting the central well with at least one inlet well and at least one outlet well.

In an aspect, the perfusable network is accessible from the top of the central well.

In an aspect, the perfusable network is extractable from the top of the central well.

In an aspect, the multi-well plate comprises 6 wells, 12 wells, 24 wells, 96 wells, 384 wells, or 1536 wells.

In an aspect, the multi-well plate comprises of 2, 4, 8, 32, 128, or 512 central wells.

In an aspect, the perfusable network mimics a blood or lymph vessel network, the architecture of an organ or a tissue, or a cavity of an organ or a tissue.

In an aspect, the seeded cells comprise hepatocytes, pancreatic Islet cells, fibroblasts, chondrocytes, osteoblasts, endothelial cells, exocrine cells, smooth or skeletal muscle cells, myocytes, adipocytes, ectodermal cells, ductile cells, kidney cells, intestinal cells, parathyroid cells, thyroid cells, nerve cells, ocular cells, integumentary cells, pluripotent cells, stem cells, or combinations thereof.

In an aspect, the extracellular matrix is a hydrogel.

In an aspect, the extracellular matrix comprises collagen, fibrin, fibrinogen, basement membrane proteins, gelatinous protein mixture secreted by Engelbreth-Holm-Swarm (EHS) mouse sarcoma cells or a combination thereof.

In an aspect, the multi-well plate further comprises spheroids, organoids, or a combination thereof embedded within the perfusable network.

In an aspect, the perfusable network is for use in vitro for research and development.

In an aspect, the perfusable network is for use in vivo for cell therapy.

In accordance with an aspect, there is provided a method for constructing a perfusable network of self-assembled cells, the method comprising:

combining a plurality of cells and a gel matrix into a mixture,

transferring the mixture into the central well of the multi-well plate described herein, and

allowing the plurality of cells to self-assemble into a perfusable network.

In an aspect, the method further comprises transferring spheroids, organoids, or a combination thereof into the central well with the mixture in step b).

In an aspect, the method further comprises removing the perfusable network from the central culture well of the cell culture device.

In accordance with an aspect, there is provided a perfusable network of self-assembled cells constructed using the method described herein, wherein the network mimics a blood or lymph vessel network, the architecture of an organ or a tissue, or a cavity of an organ or a tissue.

In an aspect, the perfusable network further comprises spheroids, organoids, or a combination thereof embedded within the network.

In an aspect, the network is for use in vitro for research and development.

In an aspect, the network is for use in vivo for cell therapy.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1 shows a step-by-step schematic of the cell culture device setup and operation in an exemplary embodiment of the disclosure: a) cell culture device filled with different colored dyes to depict the 128-independent units—the microfluidic device is fabricated by introducing microchannels to a customizable 384-well plate with 3 wells (inlet, tissue well and outlet) together making one perfusable unit; these perfusable microchannels are shown enlarged above for better visualization; b) illustration of the cell culture device and experimental set up for vascularization of colon organoids cultured in the cell culture device; the matrices containing colon organoids, endothelial cells and fibroblasts are casted on to the bottom of the tissue well and, after gelation, media is perfused into the vascular network by placing the plate on a programmable rocker; c) illustration of 96-well, 24-well, and 12-well plate versions of the cell culture device; d) illustration of a perfusable microvasculature bed within a hydrogel matrix in the tissue well of the cell culture device.

FIG. 2 shows a schematic of the cell culture device operation and interstitial flow in hydrogel in exemplary embodiments of the disclosure: a-b) image and illustration of the cell culture device containing an array of 3-well perfusion units connected with micro-channels (scale bar is 3 mm); c) illustration of vascularized hepatic spheroids in the cell culture device; d) distribution of color dye in fibrin gel over time under either interstitial flow or passive diffusion in the cell culture device (scale bar is 3 mm; white dotted lines outline the edge of the well).

FIG. 3 shows the culture of a perfusable microvascular bed in the cell culture device in an exemplary embodiment of the disclosure: a) fluorescent images of GFP-HUVECs assemble into a microvascular network over time with or without the presence of fibroblasts (scale bar is 2 mm; white dotted lines outline the edge of the well); b) confocal image of the microvascular network with or without fibroblasts on day 5 stained for F-actin, GFP, and DAPI (scale bar is 200 μm); c) high magnification fluorescent images of the microvascular network perfused with the dextran over time showing perfusion of fluorescent 70 kDa dextran (green) from the inlet well, thorough the microvascular bed, to the outlet well (scale bar is 3 mm, white arrow indicates flow direction).

FIG. 4 shows fluorescent images of GFP-endothelial cells which self-assemble into microvascular networks at different initial cell seeding densities over a period of time in an exemplary embodiment of the disclosure (scale bar is 3 mm).

FIG. 5 shows the angiogenesis assay on the cell culture device in an exemplary embodiment of the disclosure: a) illustration of experimental setup for the angiogenesis assay; b) fluorescent image of GFP-endothelial cell sprouting into fibrin gel with or without fibroblasts (FBs) (scale bar is 100 μm); c) Quantification of the length of vessel sprouts in fibrin gel with or without fibroblasts (NS indicates no sprouting; * indicates p<0.05; n=3).

FIG. 6 shows the culture of vascularized liver spheroids on the cell culture device in an exemplary embodiment of the disclosure: a) brightfield and fluorescent images of liver spheroids cultured in micro-wells over 2 days prior to seeding into the cell culture device (scale bar is 500 μm; hepatic spheroids containing hepatocytes labeled with red cells tracker; GFP-endothelial cells and fibroblasts); b) brightfield images of well seeded with microvasculature, fibroblasts, and liver spheroids over time (scale bar is 1 mm; white dotted lines outline the edge of the wells and the microchannel); c) fluorescent images of corresponding wells seeding with microvasculature (GFP), fibroblasts, and liver spheroids over time; liver spheroids also contain GFP-endothelial cells (scale bar is 1 mm; high magnification images were derived from areas labeled with dotted white boxes and white arrows label the micro-vessels that penetrate the liver spheroid); d) TEM of a cross-section of a self-assembled micro-capillary vessel (scale bar is 5 μm); e) perfusion of fluorescent 70 kDa dextran through the microvascular network around the hepatic spheroid, indicating a perfusable vascular network is established in proximity to the liver cells.

FIG. 7 shows nanoparticle and nutrient delivery to liver spheroids in the cell culture device in an exemplary embodiment of the disclosure: a) illustration of intravascular nanoparticle deliver to liver spheroids; b) brightfield and fluorescent time lapse images of nanoparticle (1 μm) perfused from the vasculature and then leaked into the liver spheroid (scale bar is 200 μm; dashed arrows label a particle exiting the vessel and entering the liver spheroid; white arrows label a cluster of particles accumulating inside the spheroid); c) fluorescent images of wells seeding with microvasculature (GFP), fibroblasts, and different number of liver spheroids (labeled with celltracker red) on day 6 (scale bar is 1 mm, liver spheroids also contain GFP-endothelial cells); d) quantification of albumin secretion from spheroids cultured with or without vasculature and perfusion (n=4; * indicates p<0.05); e) image of extracted vascularized hepatic tissue that can be used for implantation (ruler shown is in mm scale).

FIG. 8 shows a culture of the perfusable microvascular bed on the cell culture device and the optimization of hydrogel matrices in an exemplary embodiment of the disclosure: a) illustration of the experimental setup; b) fluorescent images of GFP-HUVECs assemble into a microvascular network over time in the presence of fibroblasts (scale bar is 1 mm); c) confocal fluorescent images of the self-assembled microvasculature network in matrices of different formulation on the cell culture device wherein cells are stained for F-actin and DAPI; d) time-lapsed perfusion of fluorescent 70 kDa dextran from the inlet well, through the microvascular bed used for permeability quantification, to the outlet well on Day 6 (scale bar is 1 mm); high magnification fluorescent images of the microvascular network perfused with the dextran over time indicates minimal dextran leakage from the networks; e) quantification of vessel diffusion permeability in different regions of the network (n=5, ns indicates not statistically significant); f) fluorescent images of vessels stained for CD31, vWF, Laminin, GPF, and DAPI (white arrows indicate vWF fiber; scale bar is 100 μm); g) transmission electron microscope (TEM) image of the cross-section of a single vessel (scale bar is 5 μm).

FIG. 9 shows brightfield images of dissociation, passaging, and growth of human colon organoids in pure Matrigel™ (scale bar is 100 μm) in exemplary embodiments of the disclosure.

FIG. 10 shows the optimization of thrombin concentration in fibrin and fibrin/Matrigel formulation for microvasculature assembly in the cell culture system (scale bar is 1 mm) in exemplary embodiments of the disclosure.

FIG. 11 shows consistency in vascular perfusion using fluorescent images of vasculatures perfused with 70 kDa dextran over time in multiple wells of the cell culture device (scale bar is 1 mm) in in exemplary embodiments of the disclosure.

FIG. 12 shows shear stress distribution in the vascular network in exemplary embodiments of the disclosure: a) fluorescent image of the vascular network perfused with red fluorescent particles (1 um in diameter); b) quantification of shear stress and shear stress distribution by tracking particle perfusion in the vascular networks—the histogram was produced by analyzing 18 regions from 6 different wells.

FIG. 13 shows the optimization of hydrogel matrices for culturing human colon organoids in an exemplary embodiment of the disclosure: a) illustration of the experimental setup; b) brightfield images of colon organoids growing in matrices of different formulation over time in a 384-well plate (scale bar is 100 μm); c) confocal fluorescent image of a colon organoid grown in fibrin gel with 10% (v/v) Matrigel® for 8 days on static condition (stained with F-actin and DAPI); d) the quantification of organoid area in different hydrogel matrices was done using Image J (n=15 organoids) with statistical significance determined using one-way ANOVA on the ranks with Tukey's test (* indicates p<0.05; ** indicates p<0.01; *** indicates p<0.001).

FIG. 14 shows the media formulation optimization of vascularized human colon organoids on the cell culture device in an exemplary embodiment of the disclosure: a) illustration of the experimental setup b) table listing the different media conditions tested for optimizing the growth of vascularized organoids; c) brightfield-GFP overlay images of vascularized colon organoids in different media formulations are shown, again magnified on the right with fluorescence only images to better visualize the assembled tissue (self-assembled microvascular network, green; vascularized colon organoid, brightfield-GFP overlay; scale bar is 1 mm; white dotted lines outline the edge of the wells and the microchannel); d-g) quantification of characteristic features of assembled vascular network in different media formulations using AngioTool and Image J (significance determined using one-way ANOVA on ranks with Dunn's method; day 4, n=3-5); h) quantification of organoid area in different media formulations; e,f,h) statistical significance was determined using one-way ANOVA with the Holm-Sidak method (*indicates p<0.05; ** indicates p<0.01; ***indicates; p<0.001).

FIG. 15 shows a culture of vascularized human colon organoids on the cell culture device in an exemplary embodiment of the disclosure: a) brightfield images of vascularized colon organoid overlaid with fluorescent images of self-assembled microvascular network from GFP endothelial cells in ECGM2:Colon media (1:1) at Day 13 (scale bar is 1 mm; white dotted lines outline the edge of the wells and the microchannel); b) GFP-brightfield overlay image of vascularized colon organoid perfused with RFP-particle (1 μm) in the cell culture device on the left and fluorescent image only on the right (white dotted circle labels the organoid; scale bar is 250 μm); c) confocal fluorescent z-stack image of vascularized organoids stained for F-actin, DAPI and GFP-endothelial cells (inset shows the cross-section of a colon organoid); d) quantification of the dynamic assembly process of vascularized organoids over 11 days (n=6); e) histological section of normal human colon tissues, vascularized colon organoids and non-vascularized colon organoids stained for hematoxylin (nuclei), eosin (ECM and cytoplasm), E-cadherin, CD31, Villin, and Ki67 after being picked out of a well (as shown in bottom left corner) on day 11 (scale bar is 100 μm); f) quantification of the distance between the vessels and the colon epithelium in both vascularized colon organoids (vOrganoids) and human colon tissues (hColon) −19% and 28% of vessels counted were in direct contact (a distance equal to 0) with the epithelium in human colon tissue and vascularized organoids, respectively (n=3-5 independent samples with 69 measurements); g) brightfield images of colon organoids at Day 9 grown in the cell culture device vs. static 384-well plate with and without epithelial co-culture in a combination of fibrin gel and Matrigel®; h) quantification of percentage of colon organoids recovered in the four conditions (n=3-5) (significance determined using one-way ANOVA with Holm-Sidak method; * indicates p<0.05 ** indicates p<0.01 *** indicates p<0.001).

FIG. 16 shows the association of vasculature and colon organoids in histological section of vascularized colon organoids stained for CD31 scale bar is 100 μm) in exemplary embodiments of the disclosure.

FIG. 17 shows a vascularized human colon organoid model of tissue inflammation with innate immunity in exemplary embodiments of the disclosure: a) illustration of experimental setup and the process of monocyte infiltration into colon organoids in response to inflammatory cytokines; b) brightfield and fluorescent images of vascularized colon organoid immediately and one day after monocyte perfusion with and without 50 ng/ml TNF-α stimulation for 12 h (dotted white lines outline the edge of the wells and the microchannel; scale bar is 1 mm); c) fluorescent images of blood vessels treated with or without TNF-α and stained for ICAM-1 and DAPI (scale bar is 1 mm); d) quantification of ICAM-1 staining (n=3); e) quantification of monocytes attachment with and without TNF-α stimulation (n=3); f) quantification of monocyte infiltration into colon organoids with and without TNF-α stimulation (statistical significance determined using one-way ANOVA followed by the Holm-Sidak method; * indicates P<0.05 ** indicates P<0.01 *** indicates P<0.001).

FIG. 18 shows compartmentalized of the cell cultures system for the study of epithelium barrier functions in exemplary embodiments of the disclosure: a) cell seeding procedure for compartmentalized cell culture system—epithelial cells form a flat monolayer on top of the hydrogel; b) illustration of tissue models with topography on the top surface of the gel to guide cell assembly and air-liquid interface for lung and skin models.

FIG. 19 shows a cell culture system with topographical guidance for blood vessel assembly in exemplary embodiments of the disclosure: a) schematic and images of connected grooves at the bottom of a well of a cell culture device; b) fluorescent images of TRITC-vascular networks formed on top of grooves in cell culture device, showing topographically guided assembly and vascular connection to inlet and outlet wells (scale bar is 2 mm).

FIG. 20 shows biochemical gradient generation in a cell culture system in exemplary embodiments of the disclosure: a) illustration of a cell culture device fabricated by introducing microchannels to a customizable 384-well plate to generate 40 independent units—seven wells (three inlet wells, one middle well, and three outlet wells) together make one perfusable unit; b) illustration for generating a gradient of growth factors/morphogens, ions, or chemical molecules to mimic controlled morphogenesis of cells in organ development; c) illustration of different gradient patterns that can be introduced in a cell culture device to mimic the physiological organ developmental process; d) fluorescent image of Scheme 1 gradient pattern generated by adding Dextran dye to the outlet wells.

### DETAILED DESCRIPTION

In aspects, the present disclosure describes a simple-to-use microphysiological cell culture system in the format of a conventional multi-well plate for fabricating, culturing, perfusing and optionally testing multiple vascularized microenvironments within an “open-top” well connected by channels to an inlet and outlet well. The formed vascular barrier guides fluid perfusion in the vascular lumen from an inlet well to an outlet well while the interstitial space outside of the vascular network can be accessed from the top of the well. Without the physical constraints of traditional microfluidic channels, micro-tissue spheroids and/or organoids of various sizes and quantities can be incorporated and vascularized by the microvascular bed formed in the well. The large array of perfusable vascular networks can be tested in a high-throughput fashion or easily extracted from the wells for use outside the cell culture device, such as for in vivo transplantation.

### I. Definitions

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “perfusable” as used herein refers to an interconnected network or series of channels through which a liquid medium can flow or circulate, for example a perfusable vascular network that provides oxygen and nutrients to a three-dimensional cell culture.

The term “vascular network” or “vascular bed” as used herein refers to a complex network of blood vessels that supplies oxygen and other nutrients to the tissue or organ.

The term “organoid” as used herein refers to complex three-dimensional structures derived from stem cells that can represent multiple cell lineages that can mimic the complex organ physiology.

The term “spheroid” as used herein refers to three-dimensional structures derived from cell lines which represents a specific tissue component depending on the source of the cell.

### II. Cell Culture Systems and Methods of the Disclosure

Disclosed herein is an open chamber for cell culture. The chamber is open and is not considered a closed system. Typically, the chamber is open at the top of the chamber, allowing access to the cells being cultured therein. A removable covering such as a lid may be included in order to reduce evaporation or a lid may be explicitly excluded from the open chamber described herein. The chamber is not sealed and is open and accessible to the operator.

For example, the chamber is typically an open cylinder. In aspects, the chamber comprises an open top and a closed bottom, where the open top and closed bottom have substantially equal diameters. The closed bottom may be an integral part of the chamber or it may be provided by means of a separate base. For example, the chamber may be provided as a well in a multi-well plate, where the plate may be open- or closed-bottomed and optionally provided with a base to enclose the bottom end of the chamber.

The chamber comprises an inlet and an outlet and is configured for non-tangential flow of fluid from the inlet to the outlet. It will be understood that the naming of the “inlet” and the “outlet” is arbitrary and that the inlet and outlet can be reversed, meaning that the inlet becomes the outlet and the outlet becomes the inlet if, for example, fluid flow is initiated by rocking a plate and the associated gravitational forces. In this case, the flow of fluid may be bidirectional and the inlet and outlet will alternate. In other cases, the flow of fluid may be unidirectional by using a rotational rocking method or by using a pump, in which case the inlet remains the inlet and the outlet remains the outlet.

It will be understood that the inlet and outlet may be of any desired shape or configuration. Typically, the inlet and/or outlet is a channel. The channel is typically about 0.5 times to about 3 times as wide as it is tall, such as about 0.5 times as wide as it is tall or about equal in width and height. In typical aspects, the channel is about 200 microns wide and from about 100 to about 200 microns tall.

In some aspects, the open chamber explicitly does not contain a sacrificial material. The use of a sacrificial material adds an undesirable extra step in the method of forming the open chamber and is unnecessary when following the methods described herein. In additional or alternative aspects, the chamber does not comprise any membranes.

It will be understood that the inlet typically leads to an inlet chamber and that the outlet leads to an outlet chamber. It is possible for the inlet and outlet chamber to be omitted, however, particularly if a pumping system is used to circulate fluid. Typically, the open chamber, the inlet chamber, and the outlet chamber are all substantially similar wells in a multi-well plate. More than one inlet and corresponding inlet chamber and/or more than one outlet and corresponding outlet chamber may be included herein. For example, the open chamber may comprise 1, 2, 3, or 4 inlets and 1, 2, 3, or 4 outlets and corresponding inlet or outlet chambers.

The open chamber described herein may include a patterned base. Any desired pattern may be used but, typically, a pattern is chosen that assists in guiding the self-assembly of the cultured cells into a three-dimensional and perfusable cell culture model. For example, the patterned base in aspects comprises connected grooves for guiding self-assembly of cultured cells.

In aspects, the open chamber includes a hydrogel matrix on the bottom surface of the open chamber, as described in more detail below. For example, the hydrogel matrix may comprise a fibrin matrix, fibrin, Matrigel, collagen I, a decellularized matrix, or a combination thereof. In aspects, the open chamber further comprises seeded cells.

Also described herein is an array of open chambers. The array typically comprises at least one open chamber and at least one connected inlet chamber and at least one connected outlet chamber. Alternatively, the array may comprise at least one open chamber and a pumping system to perfuse fluid through the cell culture. Also described herein is a multi-well plate comprising one or a plurality of the arrays. For example, a 6-well plate in aspects would comprise two arrays, each of the two arrays comprising one open chamber, one inlet chamber and one outlet chamber.

Also disclosed herein is a cell culture system for fabricating perfusable vascular networks of self-assembled cells that can be formed, contained, and perfused inside a well of a modified multi-well plate. This modification provides a large array of vascular networks that are perfusable through inlet and outlet channels connected to adjacent wells in the plate. This cell culture device provides a scalable, robust, and cost-effective manufacturing method of fabricating perfusable vascular networks, and optionally vascularize spheroids and/or organoids within the network. By growing tissues in this dynamic, vascularized, and perfusable microenvironment with an “open-top” well design, tissue spheroids and/or organoids can be matured in vitro and used for either high throughput drug screening or extracted for in vivo implantation, leading to the convergence of microphysiological systems with regenerative medicine.

Accordingly, provided herein is a cell culture system for constructing a perfusable network of self-assembled cells comprising a multi-well plate embedded with microchannels connecting a central well with at least one inlet well and at least one outlet well. The central well is useful for culturing seeded cells within an extracellular matrix, wherein the perfusable network allows perfusion through the microchannels connecting the central well with at least one inlet well and at least one outlet well.

In some embodiments, the cell culture comprises a multi-well plate. The multi-well plate may comprise any number of wells. In some embodiments, the multi-well plate comprises 6 wells, 12 wells, 24 wells, 96 wells, 384 wells, or 1536 wells. In some embodiments, the multi-well plate comprises of 2, 4, 8, 32, 128, or 512 central wells. In some embodiments, the multi-well plate is a 384-well plate. In some embodiments, the multi-well plate allows for duo-directional fluid flow to maintain perfusion. In some embodiments, the multi-well plate allows for unidirectional flow by way of gravity-driven flow. In some embodiments, the multi-well plate has a cap to cover the open-top wells. In some embodiments, the cap of the multi-well plate is modified to include an array of microfluidic pumps to recirculate media back to the inlets of the device.

In some embodiments, the perfusable network is accessible from the top of the central well. In some embodiments, the perfusable network is extractable from the top of the central well. In some embodiments, the cell culture system may be used as a high-throughput drug testing platform. In some embodiments, the system is used to improve the experimental throughputs of organ-on-a-chip and/or organoid systems. In some embodiments, the perfusable network is used in vitro for research and development. In some embodiments, the perfusable network is used in vivo for cell therapy. In some embodiments, the device allows tissue extraction for applications such as, but not limited to, downstream analysis (e.g. gene expression analysis, histopathological analysis, etc.) or implantation for regenerative therapy.

In some embodiments, the extracellular matrix is a hydrogel. In some embodiments, the extracellular matrix comprises collagen, fibrin, fibrinogen, basement membrane proteins, gelatinous protein mixture secreted by Engelbreth-Holm-Swarm (EHS) mouse sarcoma cells or a combination thereof.

In some embodiments, the seeded cells include cells such as hepatocytes, pancreatic Islet cells, fibroblasts, chondrocytes, osteoblasts, endothelial cells, exocrine cells, smooth or skeletal muscle cells, myocytes, adipocytes, ectodermal cells, ductile cells, kidney cells, intestinal cells, parathyroid cells, thyroid cells, nerve cells, ocular cells, integumentary cells, pluripotent cells and stem cells, or combinations thereof.

In some embodiments, the cell culture system facilitates tissue seeding. In some embodiments, the system further comprises spheroids, organoids, or a combination thereof embedded within the perfusable network. In some embodiments, tissue spheroids, such as hepatic spheroids, can be vascularized by growing the tissue spheroids within a perfusable vascular bed self-assembled from endothelial cells. In some embodiments, organoids, such as intestinal organoids, can be vascularized by growing the organoids around a perfusable vascular bed self-assembled from endothelial cells. In some embodiments, the perfusable network mimics a blood or lymph vessel network, the architecture of an organ or a tissue, or a cavity of an organ or a tissue. In some embodiments, the perfusable network further comprising spheroids, organoids, or a combination thereof is used in vitro for research and development and/or in vivo for cell therapy. In some embodiments, tissue spheroids and/or organoids, can be vascularized in a scalable manner and then subsequently extracted for in vivo implantation. In some embodiments, tissue spheroids and/or organoids are matured in vitro and used for either high throughput drug screening or extracted for in vivo implantation.

Also described herein is a method for making a perfusable network of self-assembled cells. The method typically comprises applying a hydrogel and cells to the open chamber described herein and culturing the cells, typically while applying interstitial fluid flow through the cells from the inlet to the outlet.

In typical aspects, the hydrogel and the cells are applied together as a mixture. In alternative aspects, the hydrogel and the cells are applied sequentially in any order. For example, the hydrogel may be applied first followed by the cells, or the cells may be applied first followed by the hydrogel.

Also provided herein is a method for constructing a perfusable network of self-assembled cells, the method comprising combining a plurality of cells and a gel matrix into a mixture, transferring the mixture into the central well of the multi-well plate of the system described herein, and allowing the plurality of cells to self-assemble into a perfusable network. In some embodiments, the method further comprises removing the perfusable network from the central culture well of the cell culture device. In some embodiments, the method further comprises transferring spheroids, organoids, or a combination thereof into the central well with the mixture combining a plurality of cells and a gel matrix.

A perfusable network of self-assembled cells typically constructed using the method described herein is also provided. Typically, the perfusable network of self-assembled cells is heterogenous and similar to a physiological tissue. In some embodiments, the perfusable network of the method described herein further comprises spheroids, organoids, or a combination thereof embedded within the network. In some embodiments, the perfusable network of the method described herein mimics a blood or lymph vessel network, the architecture of an organ or a tissue, or a cavity of an organ or a tissue. In some embodiments, the perfusable network of the method described herein is used in vitro for research and development and/or in vivo for cell therapy.

It will be understood that the design described herein has many advantages. For example, in aspects it uses interstitial flow through a hydrogel to achieve vascular connection, rather than tangential flow. In the device described herein, media flow towards the hydrogel at substantially all times. In conventional devices, media first flow along the hydrogel and then toward the hydrogel after the vascular connection is established. The design described herein simplifies the device so that only one inlet and one outlet is needed, which take 3 wells per tissue unit in a 384-well plate. In conventional devices, two inlets and two outlets are needed to achieve the same result, which uses 6-9 wells per unit in a 384-well plate. The simplified design described herein allows an increase in the throughput by 2-3 times in a 384-well plate.

In addition, the tissues constructed in the device described herein can be easily extracted from the device for downstream analysis without damaging the device. Demonstrated herein is histology sectioning on extracted tissues. This is not possible with conventional devices.

Furthermore, to form the hydrogel and liquid interface with the design herein, micro-structures (any features that is less than 300 microns, for example) are not needed. In conventional devices, micro-structures (e.g., posts, or phase guide) with a size that is equal to or less than 50 micron are needed. This makes the manufacturing much more costly.

To grow the vascular network in an open well, 15-254 of hydrogel is typically casted. But to grow a proper vascular network throughout the entire gel, large number of cells are needed. The amount of media in the well cannot support this. To solve this problem, much lower thrombin concentration in the fibrin gel was used to allow the fibrin to gel at a much slower rate so that cells have time to settle to the bottom of the well. With a high density of cells at the bottom of the well, cells can form a proper vascular network at the bottom which can be more easily visualized with an inverted microscope. This technique allows a significant reduction in the amount of cells needed for each tissue, which makes this open well design feasible for creating perfusable networks inside an open well.

Moreover, because of the innovation in the design of the plate and the use of hydrogel, users do not need to pipette or cast any gel solution that is less than 15 microliters. Some conventional devices require the user to pipette 1 microliter, which is extremely difficult to do.

Because of the simpler design, additional inlets and outlets can be added to each tissue unit to create much more complex models. Up to 4 inlets and 4 outlets can be added by using all 9 adjacent wells. For example, lymphatic vessels can be added by including just one more outlet. Growth factor gradience can also be included in the hydrogel using multiple inlets/outlets. Conventional systems cannot become more complex because they have already used up all 9 wells for each tissue unit.

It has also been demonstrated that grooves can be patterned at the bottom of the open well and these grooves may be used to guide the assembly of blood vessel networks; for example to create parallel aligned vessels with parallel grooves.

### EXAMPLES

The following non-limiting examples are illustrative of the present disclosure:

### Example 1. Multi-Well Cell Culture Device

Plate Fabrication. The cell culture device platform consists of two components: a 384-well bottom-less plate (82051-544, Greiner Bio-One), and a bottom 813-μm-thick, 7.5 mm×11.4 mm polystyrene sheet (V16013, Jerry's Artarama). To embed an array of micro-channels inside the plate, a sacrificial material was patterned, poly(ethylene glycol) dimethyl ether (PEGDM, Mₙ ˜2000, 445908-50G, Sigma-Aldrich) onto the polystyrene sheet. Using standard photolithography technique, a SU8 master mold patterned with an array of inlet and outlet channels in the format of standard 384-well plate was first fabricated. Then, a polydimethlsiloxane (PDMS, Sylgard™ 184, 4019862, The Dow Chemical Company) mold containing the patterned micro-channels was prepared with a base-to-catalyst-mix ratio of 30:1 and allowed to cure overnight at 47.5° C. to ensure complete curing while keeping the PDMS mold soft. The PDMS mold was then demolded and soaked in 5% (w/v) pluronic acid (Sigma Aldrich, Cat #P2443-250G) for 30 minutes. After washing in distilled water, the PDMS mold was then capped onto a plasma treated polystyrene sheet. Next, PEGDM pellets were melted at 65-70° C. and injected into the micro-channels with a syringe. The injected PEGDM fluid was allowed to solidify at 4-25° C., then the PDMS mold was slowly peeled off from the polystyrene sheet, leaving behind the patterned PEGDM features on the polystyrene sheet. To bond the patterned polystyrene sheet with the 384-well bottomless plate, a PDMS glue (Sylgard™ 186, 2137054, The Dow Chemical Company) was used. Before bonding, the bottom-side of the bottomless plate was plasma-treated for 90 s. 5 g of the PDMS glue was first spread onto a glass slide, and then the glass slide was used to stamp the PDMS glue onto the bottom of the bottomless plate. The polystyrene sheet containing the PEGDM features was then pressed onto the well plate to seal the bottom. The two components were held together by metal clips overnight while the PDMS cured. The metal clips were removed, and the plate was packaged in sealed plastic bags and gamma ray sterilized. Prior to use, 904 of sterile distilled water was added to each well of the plate incubated overnight at 37° C. and 5% CO2 to wash off the PEGDM features and to prime the device for cell seeding.

Alternatively, the device can be fabricated using a scalable industrial method. For example, a 384-well bottom-less plate (82051-544, Greiner Bio-One) can be machined with a CNC milling machine to create an array of open channels on the bottom side of the plate. Then the plate can be bonded to a 813-μm-thick, 7.5 mm×11.4 mm polystyrene sheet (V16013, Jerry's Artarama) with laser welding. This is a simpler manufacturing method. The resulting product is completely made of polystyrene and does not contain any PDMS material or any sacrificial materials.

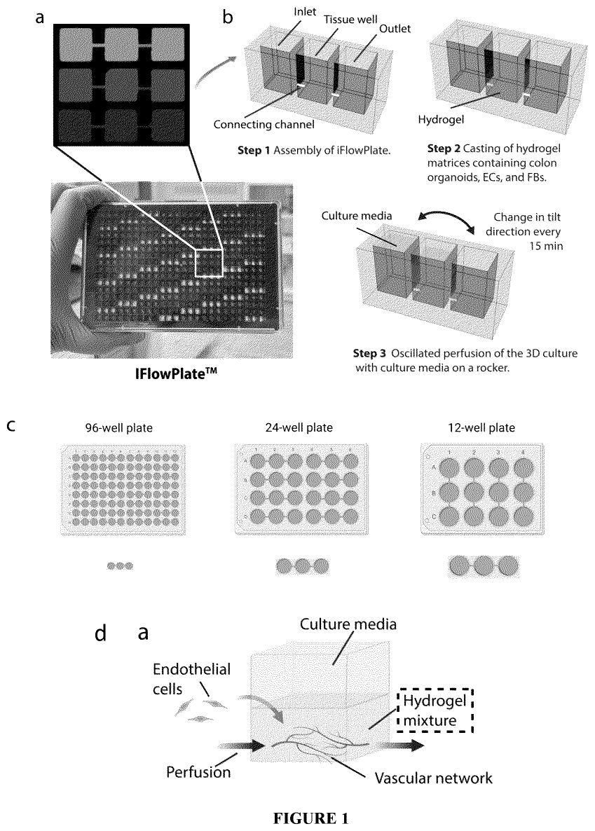

The customized standard 384-well plate thus contained added micro-channels with a cross-section of 300 μm in width and 120 μm in height at the bottom of the plate to connect 3 adjacent wells together. The 3 wells connected together become one independent unit (FIG. 1a-b). The well in the middle serves as the culture chamber where a natural hydrogel mixed with cells (and optionally, spheroids/organoids) will be casted to the bottom (FIG. 1b). The other two wells serve as the inlet and outlet media reservoir. During tissue culture, the entire plate can be placed on a programmable rocker that can tilt the plate at a 30-degree angle to produce a pressure head to drive media perfusion from the inlet well through the middle well to the outlet well. To sustain media perfusion for long periods of time, the perfusion direction may be altered by simply changing the title direction every 15 min. This configuration, without the use of any tubing or syringe pump, can maintain constant media perfusion through the gel and through the vascular network that will be established in the gel. All 128 independent units on the 384-well plate can be perfusion simultaneously. Gel casting, cell seeding, culture media changes, and any future drug tests can all be performed with simple pipetting techniques or even robotic handling systems. The device is also designed to contain minimal amounts of drug absorbing glues or PDMS materials to prevent unspecific absorption of small hydrophobic molecules. The base of the plate is made of an optically transparent polystyrene sheet of less than 1 mm in thickness to allow automatic imaging in standard plate readers and image cytometers.

The cell culture device can be adapted in different well formats and throughputs because the design principle is not constrained by the size of the well or the number of wells. The platform can be in the format of 6-well 12-well, 24-well, and 96-well plates, etc. depending on the need (FIG. 1c). For example, larger wells in the format of 6- or 12-well plates can make it easier to surgically manipulate the tissues and scale up the size of the tissue size for in vivo implantation. Smaller wells in the format of 384-well plates make the system more suitable for high-throughput experimentation while reducing cells and media usage for each tissue.

The minimalistic design of this platform simplifies the manufacturing process. For example, with industrial injection molding method, the manufacturing process would require only a simple modification to the design of the injection mold of a standard 384-well plate to include an array of short straight channels. The rest of the of manufacturing process will remain the same. For this reason, there is no need to invent new manufacturing methods which will significantly reduce the barrier and costs of translation.

### Example 2. Perfusable Self-Assembled Vasculature

### Methods

Cell Culture. Human umbilical vein endothelial cells (GFP HUVECs) tagged with green fluorescent protein were purchased from Angio-Proteomie (CAP-0001GFP). The GFP-HUVECs were cultured in endothelial cell growth medium (ECGM2, C-22011, Promo Cell). Normal human primary lung fibroblasts and hepatocellular carcinoma cells (HepG2s) were purchased from the American Type Culture Collection (ATCC, CRL-10741). The fibroblasts were cultured in a Dulbecco's modified Eagle's medium (DMEM, 319-005-CL, Wisent Bioproducts) containing 10% fetal bovine serum (FBS, 098-150, Wisent Bioproducts). The HepG2s were cultured in Eagle's minimum essential medium (EMEM, 30-2003, ATCC) containing 10% FBS. Cells between passage 3-4 were used in all experiments. To track the hepatocytes, the cells were stained with CellTracker™ Red CMTPX (C34552, Thermo Fisher Scientific) following supplier's instruction. Aggrewell™ 800 plates (STEMCELL technologies) were used to prepare the liver spheroids according to supplier's instruction. The plates were treated with an anti-adherence rinsing solution (07010, STEMCELL technologies) to prevent cell attachment. Total of 1,100 cells was added per well. The cells were allowed to form aggregates for 6 days in the Aggrewell™ 800 plate before seeding into the cell culture device. EMEM and ECGM2 media at a ratio of 1:1 (v/v) was used as the co-culture media for vascularized spheroid culture.

Gel casting and cell seeding. For gel casting, 25 uL of fibrin gel (10 mg/mL, F3879-1G, Sigma-Aldrich) with GFP-HUVECs at a seeding density of 3-5M cells/mL and thrombin (0.25 U/mL, T6884-100UN, Sigma-Aldrich) were casted in each center well. For vascularized spheroids, 20-30 spheroids were used per 254 of fibrin gel. After casting, the gel was allowed to crosslink for 25 mins at room temperature. After 25 mins, the co-culture media was supplemented with 20 μg/ml aprotinin (616370-100MG-M, Sigma-Aldrich) and added to the inlet, outlet, and center wells at a volume of 40 μL, 80 μL and 80 μL, respectively. The cell culture device was then placed on a perfusion rocker with the stage tilted at a 30° angle and the tilt direction programmed to change every 15 min. Media in inlet, outlet, and center wells were changed daily.

Functional assay. To confirm the perfusibility of the self-assembled vasculature, fluorescein isothiocyanate-dextran (2 mg/mL, average Mw 70,000, 46945-100MG-F, Sigma-Aldrich) in D-PBS was used. On day 6 after cell seeding, all the media in the wells were aspirated. 60 μl of media was added to the center well. No media was added to the outlet well, and 90 μl of the dextran solution was added to the inlet well. The vasculature was imaged immediately using a Cytation™ 5 multi-mode reader. To visualize the flow inside the vasculature and the vascularized spheroids in the cell culture device, fluorescent particles (1.0 μm, amine-modified polystyrene, L1030-1ML, Sigma-Aldrich) diluted at a ratio of 1:250 in D-PBS were added to the inlet wells. Fluorescent and brightfield videos were captured using a Cytation™ 5 multi-mode reader and a Nikon tissue culture microscope. For the angiogenesis assay, GFP-endothelial cells (0.6M cells/mL) were seeded in the inlet wells. Fibrin gels with or without fibroblasts (0.5M cells/mL) were cast in the center wells. ECGM2 media were added to all three wells and changed every day. The cell culture device was then placed on the rocker for perfusion. Fluorescent images were taken with a Cytation™ 5 multi-mode reader to track vascular sprouting every 3 days.

Immunofluorescent staining and TEM imaging. To assess the morphology of assembled vasculature, the tissue samples were fixed in 10% formalin (HT501128-4L, Sigma-Aldrich) 8 days after seeding and then blocked with 5% normal goat serum (NS02L-1 ML, Sigma-Aldrich). The tissues were then immunostained for F-actin (phalloidin-iFlour™ 594 conjugate, 20553-300, Cedarlane Labs) and DAPI (D9542-5 MG, Sigma-Aldrich) following standard procedures. Finally, the tissues were imaged with a confocal microscope (Nikon Al confocal with ECLIPSE Ti microscope). Transmission electron microscopy (TEM) images were taken to capture the morphologies of the vascular networks and the liver spheroids formed. The samples were picked out from the cell culture device using a tweezer and sections were prepared for Electron Microscopy (EM) then imaged by EM.

Albumin assay. For the static liver spheroids-only group, the liver spheroids were cultured in a standard 384-well plate without perfusion. All media were collected and changed daily. For the perfusable liver spheroids with vasculature group, liver spheroids were cultured in the cell culture device in the presence of endothelial cells. All media were collected and changed from the inlet, outlet, and center wells daily. Quantification of secreted albumin in collected culture was conducted using an Albumin Human ELISA kit (501400-96, Cayman Chemical Co) according to the manufacturer's protocol, and the data were normalized to the number of hepatocytes.

### Results

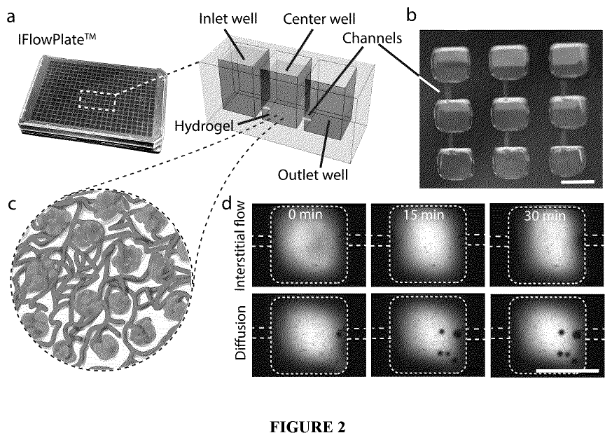

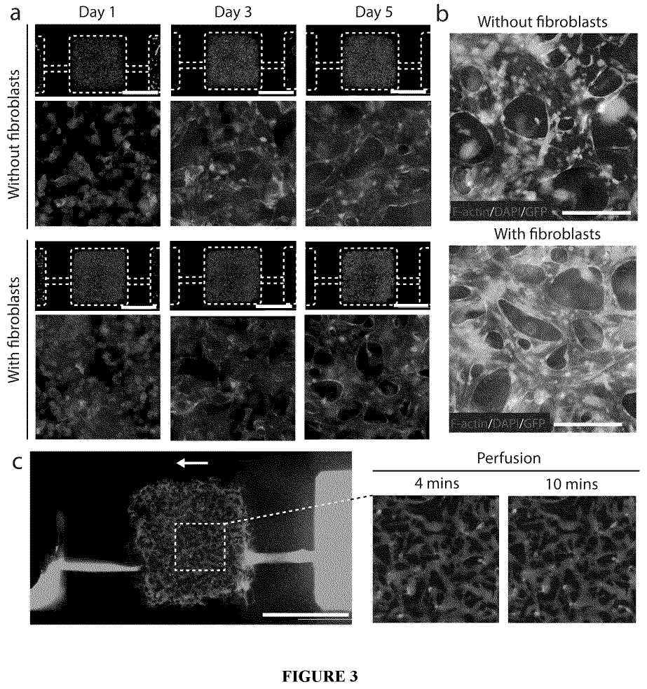

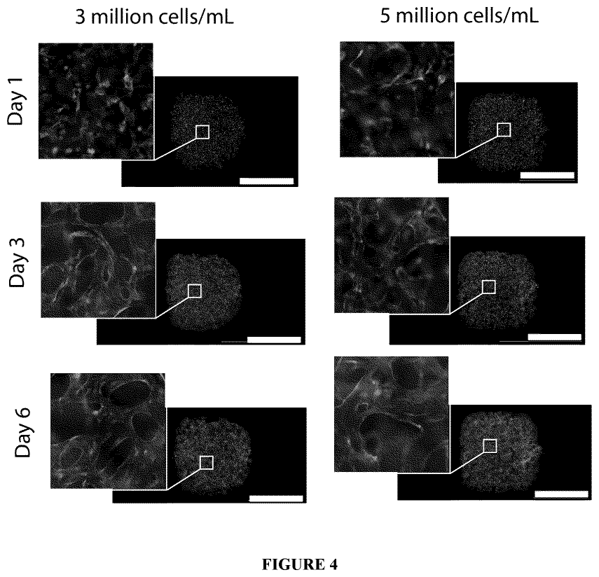

To demonstrate the feasibility of producing perfusable self-assembled vascularized cell cultures in multi-well plates, a customized 384-well plate cell culture device was formed containing embedded micro-channels connecting three adjacent wells to form an array of 128 individually perfusable units (FIG. 2a-b). Natural hydrogel, such as fibrin gel, seeded with human endothelial cells and/or fibroblasts can be cast into the center well, while the other two wells function as an inlet and outlet for perfusion (FIG. 1d and FIG. 2c). A gel-liquid interface forms in between the center well and the inlet/outlet channels. Perfusion is established with gravity by tilting the plate at a 30° angle, with the direction of the tilt being alternated every 15 min to maintain perfusion. Interstitial flow was first established through the porous fibrin gel which allows rapid mass transport through the hydrogel as shown by the diffusion of color dyes from the inlet and outlet (FIG. 2d). In comparison, without a pressure head driven by gravity, passive diffusion alone led to much slower mass transport through the hydrogel with no discernable gradient established in 30 min (FIG. 2d). When the fibrin gel was embedded with endothelial cells (GFP-human umbilical cord vein endothelial cells, GFP-HUVECs), the cells can self-assemble into a perfusable microvascular network that will connect to the inlet and outlet channels. Therefore, the initial interstitial flow through the hydrogel will be gradually redirected through the vascular network after the vascular connection is established as early as 5 days after cell seeding. It was found that the endothelial cells can self-assemble into a perfusable microvasculature, with or without the presence of fibroblasts, emulating the vasculogenesis process (FIG. 3a-b). The density of endothelial cells seeding is important to forming a connected vessel network, and a seeding density of 3-5 million/mL was found and was adequate for vessel connection (FIG. 4). The fibrin gelation time, controlled by the final concentration of thrombin in the gel mixture (0.25 U/mL), was sufficiently slow so that the gel could be cast into a large number of wells before gelation to facilitate high throughput tissue production and screening. The self-assembled vessels had a diameter between 10-80 μm, similar to native capillary vessels¹⁴. It was observed that fibroblasts were always distributed outside of the microvasculature and some wrapped around the microvasculature from the exterior vessel surface. It was determined that the addition of aprotinin (1% (v/v)) in the culture media is necessary to prevent fibrin gel degradation or compaction caused by the presence of fibroblasts, ensuring the hydrogel scaffolding stays intact in the presence of fibroblasts. At the appropriate cell seeding density, the endothelial cells are sufficiently close to each other to form an interconnected vascular network. The network can connect to inlet and outlet channels and media perfusion can be established through the entire network as show by the perfusion of 70 kDa fluorescent dextran. The resulting vascular network of endothelial cells formed a tight vascular barrier that can confine large fluorescent proteins (70 kDa dextran) in the luminal space with minimal leakage over time (FIG. 3c). Microparticles can also be perfused through the network. This perfusable microvascular bed is positioned entirely inside a well with an open top that will allow the addition of other tissue samples on top of the vascular bed as well as the extraction of the tissues from the well.

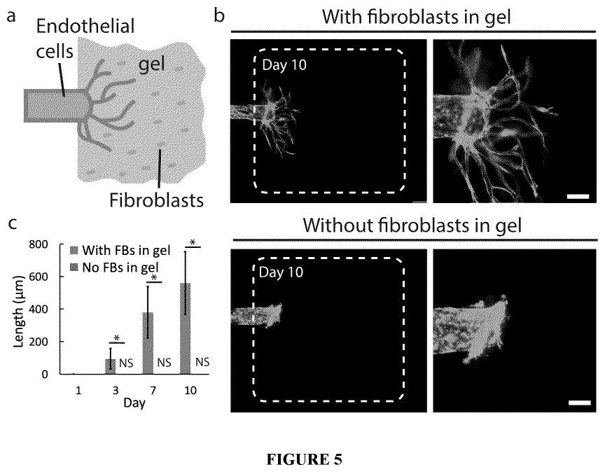

Even though the presence of fibroblasts did not make a significant difference to the vasculogenesis process, vascular sprouting in angiogenesis appears to be guided by the presence of fibroblasts. To study angiogenesis, endothelial cells can be seeded inside the inlet channel against the gel liquid interface in between the center well and the inlet channel (FIG. 5a). When fibroblasts are present inside the gel in the center well, endothelial cells were able to sprout into the gel towards the fibroblasts (FIG. 5b). However, without fibroblasts, minimal sprouting was observed, consistent with previous findings using conventional microfluidic devices which showed the presence of fibroblasts helps to facilitate angiogenic sprouting^{6,11}. The extent of sprouting can be quantified and can be used as an assay for screening pro- or anti-angiogenic drugs on this high-throughput platform (FIG. 5c).

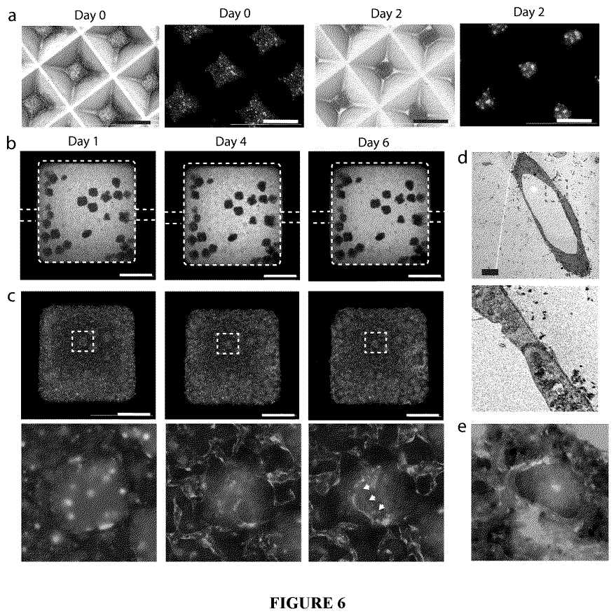

To integrate this vascular network with larger solid tissues, liver spheroids were fabricated consisting of a diameter of around 200-300 μm by aggregating hepatocytes (HepG2), endothelial cells (GFP-HUVECs), and fibroblasts in an array of funnel-shaped micro-wells (FIG. 6a). These spheroids were then mixed with endothelial cells and fibroblasts in fibrin and cast into the cell culture device. Endothelial cells inside the spheroid were able to self-assemble within the spheroid while the endothelial cells outside of the spheroids formed a microvascular network around the spheroids. In some cases, the endothelial cells were able to form microvessels which penetrated through the tissue spheroid (FIG. 6b-c). Around the spheroids, microvascular remodeling was also visible, where small changes in the vascular network structure can be seen and tracked from day to day. The vascular network around the spheroids are also perfusable, indicating that nutrients and oxygen can be delivered with convective flow directly to the spheroids after the vasculature is established on day 6 (FIG. 6e). Taking advantage of the fact that tissues can be physically extracted for downstream analysis, the extracted tissues were imaged using Transmission Electron Microscopy (TEM) and cross-sections of micro-vessels with tight intercellular junctions between the endothelial cells were found (FIG. 6d). In the application of drug testing, other downstream analyses, such as histology sectioning, transcriptome and proteomic assays, could also be performed on the extracted tissues.

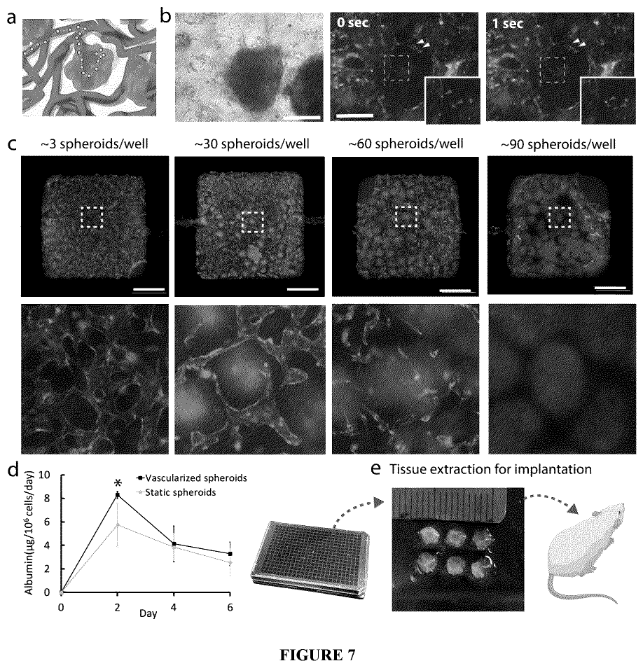

GFP-nanoparticles were also delivered through the established micro-vasculature and it was found that the particles tend to leak out from the vessels in the region around the spheroids (FIG. 7a-b). Some particles were able to enter the tissue spheroids and accumulate inside the spheroid. This delivery process can easily be visualized on the platform and could be used to model drug delivery to target tissues or tumor microenvironment. Due to the size of the well and the limited amounts of culture media in the 3-well perfusion unit, there is an upper limit on the number of tissue spheroids this system can support. It was found that at least 30 spheroids can be maintained within one well (FIG. 7c). But when over 60 spheroids are present, the lack of nutrients and oxygen led to the deterioration of the vascular network without which the rate of mass transport inside the hydrogel were slowed down even further. At a density of 90 spheroids per well, microvasculature was only visible in the peripheral of the well and failed to form in between the spheroids. Thus, 30 spheroids were applied per well as the optimized liver spheroid seeding density which could change depending on the metabolic rate of the specific tissue spheroids. Furthermore, it was found that perfusion and the presence of vasculature significantly improved the production of albumin from the hepatic spheroids on day 2 (FIG. 7d). The production of albumin plateaued after that, consistent with previous findings¹⁵. Finally, it was demonstrated that the vascularized tissues can be physically extracted from the well plate for implantation (FIG. 7e). Such pre-vascular assembly and perfusion in vitro could accelerate vascular connection and maturation, thus improving parenchymal tissue survival and post implantation in animal models.

### Discussion

A platform that allows easy integration of microvasculature with solid tissue spheroids to build complex tissues was developed. The platform is based on the conventional 384-well plate which is the gold standard in biological research, easy to use and can be operated with just simple pipetting techniques. The same platform can be applied to building other vascularized tissues such as vascularized tumor spheroids, adipose tissues, and organoids, etc. Because the luminal space of the vasculature is compartmentalized from the parenchymal space outside the vasculature, different culture media or stimuli could be applied from the center well and the inlet/outlet wells to facilitate multicellular co-culture. Furthermore, inside the 3D gel, fibroblasts and other stromal cells can also closely associate and interact with the parenchymal tissues and the microvasculature. These intercellular interactions play an important role in the study of diseases such as fibrosis, edema, and thrombosis, etc. Epithelial cells could also be added onto the hydrogel surface over the microvascular bed, and if used to model the air-liquid interface of the lung epithelium or the human skin, culture media in the center well could be removed and nutrients supplied to the epithelium layer through the underlying perfusable microvascular network. The cell culture platform disclosed herein lacks any physical barrier such as a synthetic membrane, allowing for seamless vascular integration with incorporated epithelium and parenchymal tissues.

A feature of this system is that the entire tissue, including the microvasculature, can be extracted from the center well in its entirety for downstream analyses, as well as for implantation. This allows for the tissue to first be matured in vitro under vascular perfusion and then implanted in vivo. Implantation of an already perfused microvascular network that ensures the connectivity of all vessels could accelerate vascular integration and perfusion in vivo. Accelerating vascular connection upon tissue implantation is critically important to ensuring tissue survival in regenerative medicine¹⁶. Previously, patterned vasculature has been shown to improve vascular integration in vivo but have not been pre-perfused in vitro prior to implantation¹⁷ Other systems that allow the culture of perfusable microvasculature in vitro do not allow for easy tissue extraction for implantation⁸. This is the first system that allows in vitro microvascular perfusion followed by the complete removal of the vascularized tissue for implantation on a scalable platform. The ability to produce large quantities of these vascularized tissues in the format of a 384-well plate is another important feature that could find application in high-throughput drug discovery or regenerative therapy with a modular tissue engineering approach¹⁸.

The fibrin-based hydrogel matrix could be replaced with organ-specific decellularized matrices¹⁹. When combined with organ-specific endothelial cells, a more organ-specific microvascular environment could be established²⁰. Inclusion of stromal cells, such as pericytes or smooth muscle cells, in addition to fibroblasts, will also further enhance vessel maturation and stability. To improve the liver model, Kupffer cells that are responsible for regulating inflammation and fibrosis could be included in the liver spheroids in disease modeling²¹. The current design relies on duo-directional fluid flow to maintain perfusion. However, if unidirectional flow is needed, the platform could be adapted based on recently published strategy that can achieve unidirectional perfusion with gravity-driven flow²².

### Example 3. Formation of Vascularized Organoids

### Methods

Endothelial and stromal cell culture. Green fluorescence protein-tagged human umbilical vein endothelial cells (GFP-HUVEC), and primary human lung fibroblasts were both purchased from Cedarlane labs (Cat #CAP-0001GFP, PCS-201-013). GFP-HUVECs were cultured in Endothelial Cell Growth Media (ECGM2, Cat #C-22011) as instructed by the supplier (Cedarlane labs). Primary human lung fibroblasts were cultured in DMEM media supplemented with 10% fetal bovine serum (FBS), 1% penicillin-streptomycin solution (100X) and 1% HEPES (1M). Cells used for all the experiments are between passage 2-5. Prior to cell seeding, all cells were strained through 40 μm cell strainers to get a single cell suspension.

Organoid culture and expansion. Colorectal organoids were acquired from the University Health Network (UHN) Princess Margaret Living Biobank in Toronto, Canada. The use of patient-derived organoids was approved by Hamilton Integrated Research Ethics Board. The colon organoids were cultured in Intesticult™ human organoid growth media purchased from Stemcell Technologies (Cat #06010) according to the manufacturer's protocol. Specifically, frozen vials of organoids were thawed and embedded in growth-factor reduced Matrigel® (Corning, Cat #CACB356231). Each vial of organoid was mixed with 150 μL of Matrigel®. The organoids were cultured in a regular 24-well plate with each well containing 50 μL of Matrigel®-Organoid mixture in the center of the well (Matrigel® dome). Organoids were cultured for 1 week until they fully recover and later passaged. Organoids were dissociated and expanded with two different methods. For the mechanical dissociation method, the Matrigel® containing the organoids were dislodged from the wells and collected in a tube. The Matrigel® was then broken into small fragments by repeated pipetting using a fire-polished Pasteur pipette. To this mixture, fresh Matrigel® was added and plated into a new 24-well plate. For the TrypLE method, the Matrigel® was first degraded by incubating the organoids with 1 mL of Cell Recovery Solution (Corning, Cat #CACB354253) per each well on ice for 1 hour. To this mixture, 5 mL of cold Advanced DMEM/F12 media (Gibco, Cat #12634-010) was added and centrifuged at 200G for 4 minutes. The supernatant containing the Matrigel® fragments were discarded, and the organoids were incubated in a water bath (37° C.) for 10 minutes with the 1 mL of TrypLE™ express enzyme (Gibco, Cat #12605-010). This mixture is then centrifuged again at 200G for 4 minutes and fresh Matrigel® was added after discarding the supernatant. For both methods, organoids from each well were expanded into three wells, or a splitting ratio of 1:3. The organoids were cultured for a week and the media were changed every 2 days. The organoids expanded using both methods were cryopreserved by breaking the hydrogel into small fragments and collected using the Recovery™ cell culture freezing media (Gibco, Cat #12648-010). For all experiments in the cell culture device, organoids were thawed from the frozen vials and applied directly to the cell culture device. The colorectal organoids used for all the experiments were between passage 13-17.

Hydrogel formulation. The hydrogel matrices for the cell culture device cell seeding were prepared by mixing 10 mg/mL fibrinogen with 10% (v/v) Matrigel®. The hydrogel mixtures were aliquoted into 125 μL aliquots. To this 125 μL gel aliquot, 25 μL of thrombin (1.5 U/mL) was added and mixed prior to casting. 25 μL of this final mixture was then casted into each well. In general, three wells were cast at a time. For the ECM optimization experiments, 5 mg/mL fibrin, 10 mg/mL fibrin and pure Matrigel® were also used. Fibrinogen and thrombin were purchased from Sigma Aldrich (Cat #F3879-1G, T6884-100UN) and stock solutions were prepared as per manufacturer's instructions and stored at −20° C. For experiments with static conditions, colon organoids were suspended in pure Matrigel® and cultured in colon media within a regular 384-well plate with no perfusion.

Cell seeding and device operation. The cell culture device was sterilized and first incubated with sterile distilled water to dissolve the PEGDM inside the plate and prime the plate overnight at 37° C. After overnight incubation, the plate was then centrifuged at 40G for 30 seconds to remove any air bubbles inside the plate. The wells were then washed with sterile water again to remove any residual PEGDM. HUVEC (5 million cells/mL), Fibroblasts (1 million cells/mL), and colon organoids (10-12 organoids/well) were suspended in the hydrogel mixture according to the hydrogel formulation. 25 μL of this gel mixture was then added to the corresponding tissue wells. The plate was gently tapped to allow the gel to fall to the bottom of the plate. The plate was then incubated at 37° C. for 30 minutes to allow gelation. To prevent the gel from entering the inlet and outlet channels, 25 μL of fibrin gel (10 mg/ml fibrinogen with 10 U/ml Thrombin) may be applied to the inlet and outlet well prior to casting gel in the center well. After casting in the center well, the gels in the inlet and outlet well can be aspirated and removed. Endothelial cells (0.6 million cells/ml) were also seeded in the inlet and outlet wells. The plate was maintained under the static condition to allow the cells to attach overnight. Culture media were changed in all wells the following day and the plate was placed on a programmable rocker that tilts at a 30° angle. The tilt direction was programmed to change every 15 min to maintain perfusion. The culture media were supplemented with 1% (v/v) Aprotinin (Sigma Aldrich, Cat #616370-100MG-M) to prevent fibrin degradation. The culture media were changed every other day. For optimizing vascular network formation and organoid culture, ECGM2 media was tested, colon organoid media and their mixture 1:1 or 1:9 (v/v) ratio accordingly. Mixture of ECGM2 and colon organoid media at a ratio of 1:1 was found to be the optimal media condition for the culture of vascularized colon organoids.

Immunofluorescent staining and Histology. The entire immunostaining procedure was done in the cell culture device under perfusion on a programmable rocker. Cultured tissues in the cell culture device were first washed with 1×PBS to remove residual media. The tissue was then fixed overnight under perfusion in 4° C. with 10% Formalin solution. The next day, the fixative was removed, and the tissue was washed again with 1×PBS three times and blocked for 2 hours under perfusion at room temperature with 5% normal goat-serum (Sigma Aldrich, Cat #NS02L-1ML) containing 0.1% Triton-X. The tissue was then stained with primary antibodies, Anti-CD31 (Abcam, Cat #ab28364), Anti-vWF (Abcam, Cat #ab6994), Anti-Laminin (Abcam, Cat #ab11575) overnight at 4° C. under perfusion. The following day, tissues were washed with PBS. The anti-rabbit secondary antibody (Abcam, Cat #ab150077) or F-actin conjugate antibody (Cedarlane Labs, Cat #20553-300) was added along with DAPI (Sigma Aldrich, Cat #D9542-5MG) and incubated at room temperature for 2 hours under perfusion. After incubation, the samples were washed in PBS overnight and imaged using a confocal microscope (Nikon SR-SIM) or image cytometer (Biotek Instruments). All the antibodies were diluted at 1:100 ratio in PBS with 2% (v/v) FBS. For histology, the cultured tissues were fixed in 10% formalin solution for 48 hours. The tissue was then removed from the well using a tweezer and was placed in histology cassettes. The cassettes were then immersed in 70% ethanol until ready for paraffin wax embedding. The embedded tissues were then sectioned and stained with hematoxylin and eosin, E-Cadherin (Abcam, Cat #ab1416), CD31 (Abcam, Cat #ab28364), Villin (Abcam, Cat #ab130751) and Ki67 (Abcam, Cat #16667). The human colon tissue sections used for histological analyses were a generous gift from the John Mayberry Histology Facility at McMaster University. For imaging vascular network using Transmission Electron Microscopy (TEM), the tissue was fixed for 1 hour in 1% osmium tetroxide in 0.1M PBS. The fixed samples were then immersed in a series of ethanol dilutions (50%, 70%, 70%, 95%, 95%, 100%, and 100%) to dehydrate the sample. The dehydrated sample was then embedded in 100% Spur's resin and was allowed to polymerize overnight. The embedded tissue was then sectioned and stained with uranyl acetate and lead citrate before imaging.

Tissue quantification and Perfusion studies. The increase in size of the colon organoids was quantified in different matrices from six wells per condition. For each well, five independent growing organoids were counted and averaged. The organoid area on Day 8 was then normalized against Day 1 values for each condition. The average diameter of the vascular networks was also quantified similarly where the vascular diameters at ten different positions were measured per well. At least 3 different wells were used per experimental group. Both the area of organoids and the vascular network diameters were quantified using Image J. The AngioTool software was used to quantify the vessel area, junction density, average vessel length of the vascularized colon organoids in different media conditions (n=3-5). The organoid growth and the vessel junction density were measured over time (11 days) to quantify the assembly process of vascularized organoids in our platform using Image J and AngioTool (n=6 wells). The percentage of organoids recovered in the cell culture device were quantified vs static condition (colon organoids only) with and without vascular network. This was done by quantifying the organoids and cellular clusters in at least 3 different wells per condition. Using Image J, the distance between the colon epithelium and the nearest vessel were also quantified in both human colon tissue (n=5 samples and 69 measurements) and vascularized colon organoids (n=3 wells and 69 measurements).

To study the perfusion through the self-assembled vascular networks, a 70 kDa TRITC-labelled dextran was used (Sigma Aldrich, Cat #T1162-100MG). In this experiment, 90 μL of TRITC-labelled dextran (500 μg/mL) was added to PBS which was then added to the inlet well and 60 μL of PBS added to the tissue well. The perfusion of the dextran molecules through the vascular network was then imaged using an image cytometer (BioTek Instruments Inc.). Time-lapse images of perfusion in 15-minute time intervals were captured. From the time-lapse images, diffusive permeability, Pa at the edge and center of wells (n=5) were calculated using the equation:

$${P}_{d} = \frac{1}{{I}_{i} - {I}_{b}} ( \frac{{I}_{f} - {I}_{i}}{Δ t} ) ⨯ \frac{d}{4}$$

Here, Iᵢ and I_{f} represents the average intensities at final and initial timepoint while I_{b} represents the average background intensities. Δt is the time interval between the images and d is the average diameter of the vessel in the chosen ROI.

To calculate the shear stress of the vascular network, the network was perfused with red fluorescent particles (1 μm in diameter). The microscope was tilted at 30-degree angle to mimic the programmable rocker. Videos of perfusion were taken at 13.13 frames/sec and the exposure time was set at 700 μs. For calculating the shear stress at different locations, the instantaneous velocity and the vessel diameter were used. The instantaneous velocity was calculated by tracing the positions of a particle in two adjacent frames and the vessel diameter was measured in Image J. Shear stress was calculated using the formula:

$$τ = \frac{4 Q ⨯ η}{π {r}^{3}} .$$

The shear stress was calculated at 18 different regions in 6 wells. To demonstrate perfusion in vascularized organoids, the vascular networks were perfused with 90 μl of red fluorescent particles in the inlet and 60 μl of PBS in the outlet. The delivery of particles to the organoids through the vascular network was then imaged using an image cytometer.

To model colon inflammation in the platform, the tissue was first stimulated by supplementing culture media 1 with 50 ng/ml of TNF-α incubated at 37° C. for 12 hours (n=3). For the no treatment group (n=3), media was not supplemented with TNF-α. After incubation, the tissues were washed and THP-1 monocyte cells (Cedarlane Labs, Cat #TIB-202) at 0.3 million cells/mL concentration, labeled using red cell tracker (Thermo Fisher Cat #C34552), were perfused from the inlet wells. 90 μL of cell suspension was added to the inlet and 60 μL of culture media to the tissue well to allow gravity-driven perfusion. The platform was perfused at 37° C. for 30 minutes. Next, the networks were washed with culture media to remove unattached monocytes and the platform was incubated at 37° C. overnight under perfusion. The monocyte attachment and organoid infiltration at Day 0 and Day 1 were imaged using the image cytometer. From these images, the monocyte attachment and percentage of organoid infiltration were quantified using Image J. To quantify ICAM-1 expression, the treated and non-treated vascularized organoid tissues were stained with Anti-ICAM-1 (Abcam, Cat #ab2213) and used Image J to quantify the percentage of stained area in the entire tissue well.

### Results

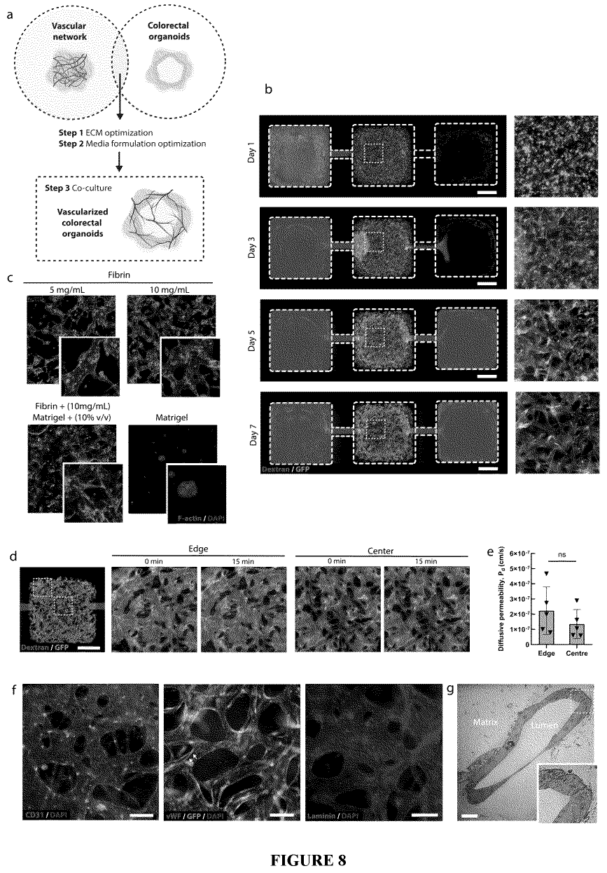

Human primary endothelial cells mixed with human fibroblasts can self-assemble into a perfusable microvascular network inside a customized well-plate in 3 days using a fibrin gel, showing this self-assembly capability is not limited to a closed microfluidic environment (FIG. 8a). The self-assembled vascular network can cover the entire well. At a cell seeding density of about 5 million cells/mL, the endothelial cells were sufficiently close to each other to form an interconnected vascular network. To sustain the generated vascularized colorectal organoids using this platform, the extracellular matrix (ECM) and media conditions were optimized (FIG. 8b).

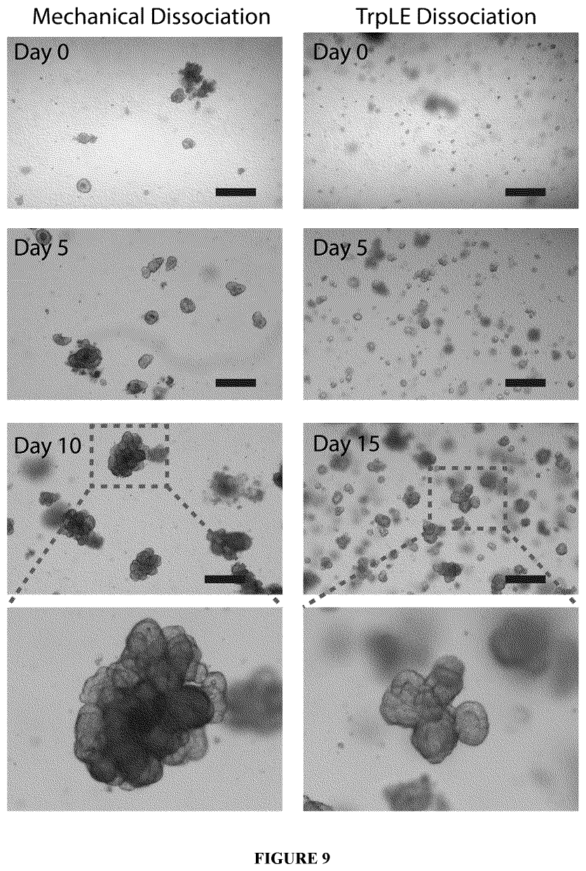

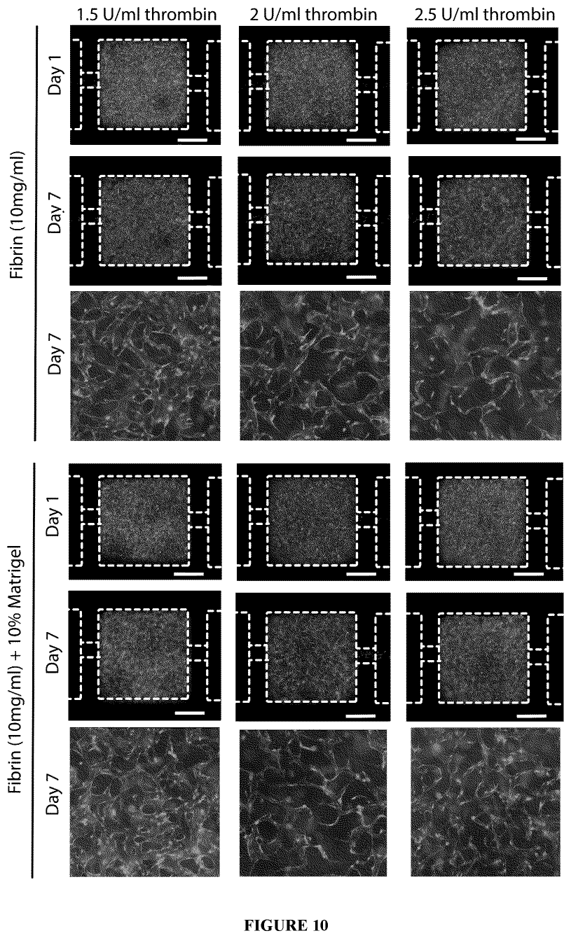

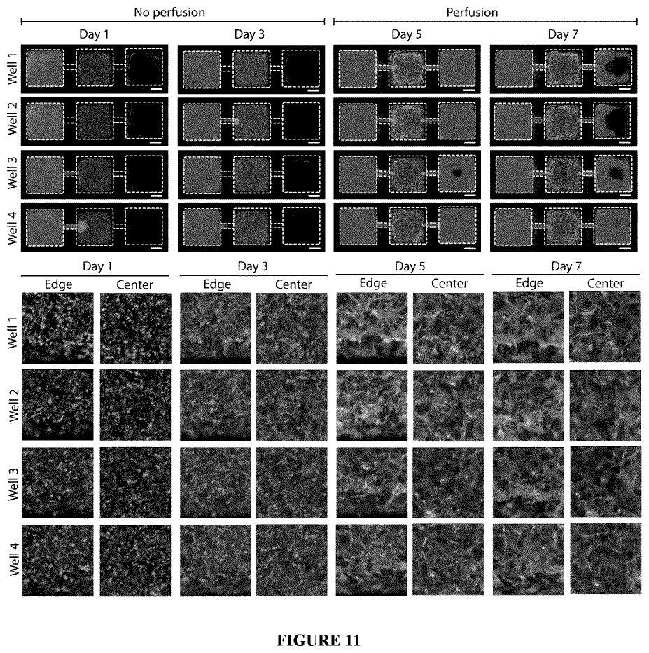

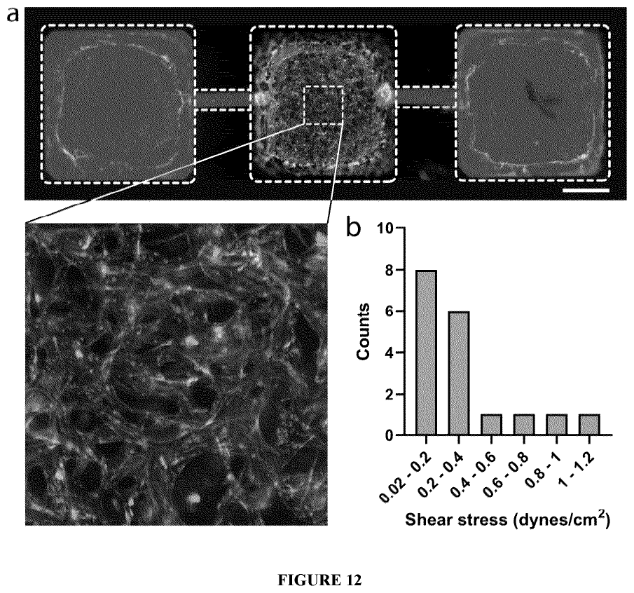

Formation of a self-assembled vasculature network can take place in fibrin gel with a fibrinogen concentration of 5 and 10 mg/mL (FIG. 8c, top). However, organoid culture usually requires the use Matrigel® which contains large quantities of laminin and collagen IV that are the building blocks of the basement membrane that supports the organoid epithelium. For example, primary colon tissue contains functional adult stem cells that reside in the base of the crypt, thus, biopsied resident intestine stem cells can undergo differentiation in Matrigel® to recapitulate the cellular diversity of the intestine epithelium (FIG. 9). Laminin and collagen IV are completely absent in fibrin gel. However, in Matrigel® alone, it was found that endothelial cells were unable to self-assemble into a vascular network (FIG. 8c, bottom). This is likely because endothelial cell alone cannot easily remodel laminin and collagen IV matrices. Therefore, to compromising between endothelial cells and organoid cultures, 10% (v/v) Matrigel® in fibrin gel was utilized and it was demonstrated that vascular assembly from endothelial cells can take place in this gel formulation (FIG. 8c, bottom). It was also found that thrombin concentration is important for the self-assembly of endothelial cells in fibrin matrices. Lower concentration of thrombin (1.5 U/ml) allowed more cells to settle at the bottom of the well, thereby facilitating the formation of a continuous vascular network (FIG. 10). The network can connect to inlet and outlet channels and media perfusion was established through the entire network on day 5, as shown by the perfusion of 70 kDa fluorescently labelled dextran (FIG. 8d). The perfusability of the network was highly consistent between different wells (FIG. 11). The vascular network formed a tight vascular barrier that can contain large proteins. Although vascular perfusion appears to be faster in vessels near the well edges due to lower flow resistance, vascular permeabilities are similar in all regions, indicating that vessels were not leaky and perfusates did not leak or pool in the center well (FIG. 8d-e). Due to the self-assembly nature of this system, the shear stress within the vascular network can vary from 0.02 to 1.2 dynes/cm² (FIG. 12). However, this level of heterogeneity is expected and resembles native tissues. The vessels formed intercellular junctions, secreted Von Willebrand Factors (vWF) important to thrombogenicity, and deposited laminin-rich basement membranes (FIG. 8f-g). Specifically, it was found that the formation of vWF fiber along the flow direction is consistent with previous reports of flow-driven assembly of vWF fiber. This self-assembled perfusable microvascular bed is positioned entirely inside a well with an open top that will allow the addition of other tissue samples both on top of and within the gel matrix as well as the extraction of the tissues from the well.

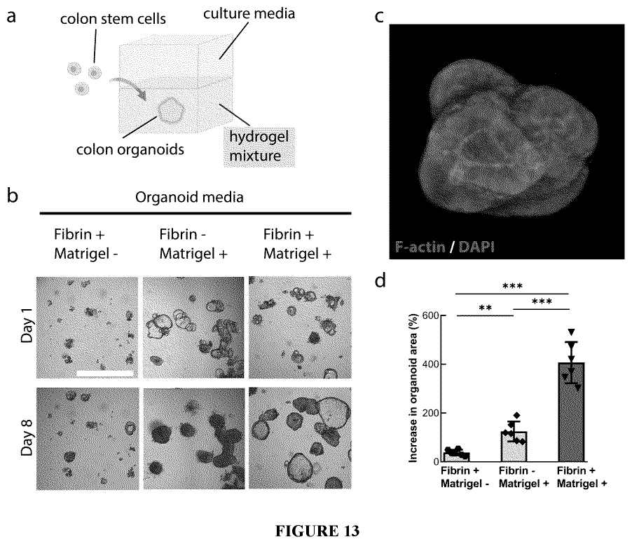

To validate the feasibility of integrating engineered microvasculature with organoids, patient-specific intestinal resident stem cells were used to grow colorectal organoids in vitro (FIG. 13a). The organoids were embedded inside the gel matrix to maximize the contact surface between the organoids and the surrounding vasculatures, and the matrix was optimized for both organoids and the vasculature. A mixture of fibrin and 10% Matrigel® was used to best support the formation of colorectal organoids (FIG. 13b). The colon organoid continued to grow for at least 8 days in and went through the crypt budding process with multiple budding structures forming. Localized staining of F-actin on the luminal surface of the organoid indicated a polarized epithelium (FIG. 13c). The cross-sectional area of the colorectal organoids was quantified in different hydrogel matrices and it was found that a combination of fibrin and Matrigel® yielded significantly larger organoids (FIG. 13d). This showed that the optimal condition for co-culture of both the engineered vascular network and colon organoids in the cell culture device would be the combination of fibrin and Matrigel®.

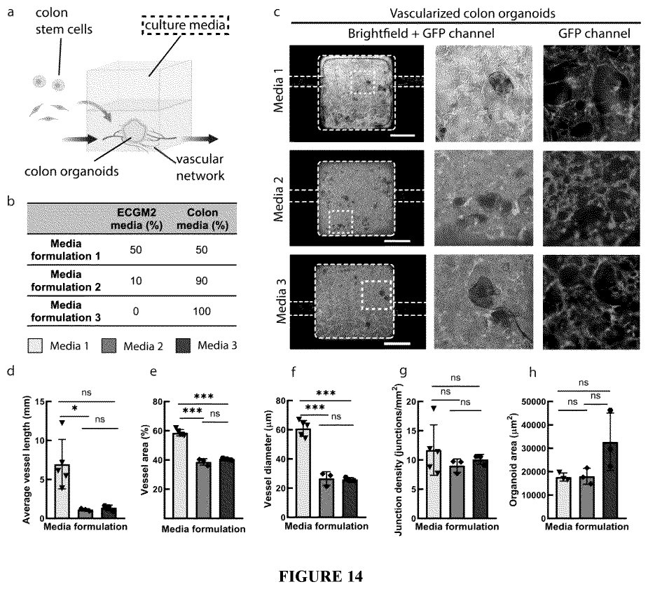

Perfusable vascularized colon organoids by co-culture of colon organoids with a pre-established microvascular bed based on the established matrix were then grown in different media conditions to find the optimal media formulation that can sustain both organoid and vascular culture (FIG. 14a,b). The vascularized organoids were then cultured in three separate media conditions: colon media; a combination of both endothelial cell growth media (ECGM2) and colon media in the ratio of 1:1; and a combination of ECGM2 and colon media in the ratio of 1:9 (FIG. 14b,c). It was found that all three media conditions were able to support organoid formation but vascular networks formed in colon media only while the 1:9 media was quite narrow. AngioTool and Image J software was used to quantify the vascularization of the colon organoids in all three media conditions (FIG. 14d-h). While the organoid area between the three media conditions showed no significant differences (p>0.05, one-way ANOVA with Holm-Sidak method), it was found that the vessel diameter and vessel area were significantly higher in ECGM2: Colon (1:1) compared to the other two conditions. The average vessel length (distance between two junctions) of 1:1 media was significantly better than 1:9 media (p<0.05, one-way ANOVA on ranks with Dunn's method) but showed no significant difference with colon media. Junctional density (junctions/area) was also quantified which showed no significant differences between the three media conditions (p>0.05, one-way ANOVA on ranks). Given that the large diameter of vascular networks formed in ECGM2:Colon (1:1) media would allow for better perfusion of the organoids and the higher percentage of vessel area observed in this condition, it was decided that 1:1 media formulation would be used for growing vascularized colon organoids in the cell culture device.

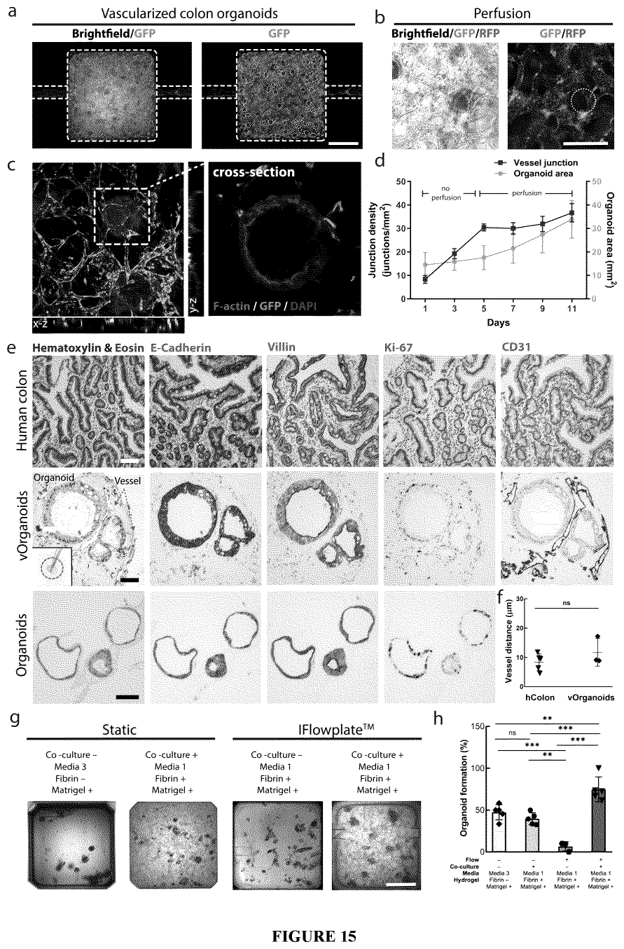

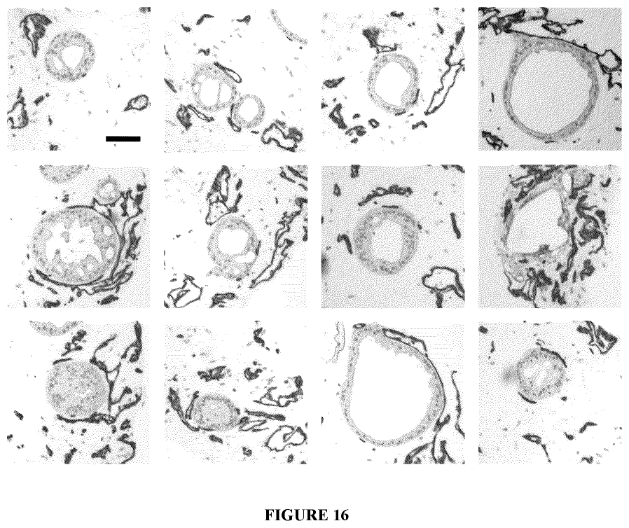

Colon organoids were cultured with a pre-established microvascular bed based on the matrix and media formulation that was established for up to 13 days (FIG. 15a). Every single organoid cultured in the well was surrounded by blood vessels (FIG. 15a). Intravascular perfusion of the engineered vascularized colon organoids was demonstrated by perfusing the vascular network with red fluorescent particles that clearly labelled the compartmentalized vascular lumen, interstitial space, and organoid lumen (FIG. 15b). Both the cytoskeleton (F-actin) and nuclei (DAPI) of the vascularized organoids were stained and imaged (FIG. 15c). Confocal images showed that the colon organoids were surrounded by GFP blood vessels in close proximity. Both organoids and the microvasculature contain hollow lumens that can be visualized from tissue cross-sections. Furthermore, polarization of the colon epithelium with clear F-actin localization on the apical side of the colon epithelium was also observed. In tracking the simultaneous self-assembly of both vasculature and organoids, it was found that the vasculature structurally stabilizes at around day 5 while the organoids grew continuously (FIG. 15d). Interestingly, accelerated growth of organoids was observed after perfusion was established on day 5. Therefore, the earliest time point for using the models would be around day 5, when the vasculature, vascular perfusion, and the organoids are well established. The open-top platform design allows the removal of the vascularized colon tissue out of the well and grants the ability to perform histological analysis (FIG. 15e). Using this feature, the vascularized organoids with human colon tissues were compared to non-vascularized organoids (FIG. 15e). Specifically, villin staining showed the organoids were polarized and expressing micro-villi. Ki67 showed the presence of proliferating progenitor cells that fueled the growth of the organoids. More interestingly, compared to the non-vascularized organoids, the vascularized organoids were surrounded with perfusable blood vessels in very close proximities similar to the native colon tissues. The distance between the organoid and the nearby vessels were measured and compared it against native colon tissues. We saw nearly indistinguishable differences were observed (FIG. 15f, FIG. 16). Moreover, 19% and 28% of vessels counted were in direct contact (a distance equal to 0) with the epithelium in human colon tissue and vascularized organoids, respectively. To further assess co-culture for organoid growth and recovery, the percentage of recovered organoids at Day 9 in different conditions was quantified and compared in the cell culture device under dynamic perfusion (1:1 media) with and without vascular network. A static condition was also tested, wherein the colon organoids were grown in pure Matrigel in the absence of vascular network under no perfusion. The colon organoid formation was significantly more robust in the cell culture device in the presence of a vascular network and perfusion (FIG. 15g,h). Specifically, it was found that there is significant cross-talk between the vasculature and the organoids in the cell culture device as organoid growth completely stopped without the presence of vasculature while using an optimized matrix and media formulation. This highlights the importance of vasculature and perfusion in supporting organoid growth and development.

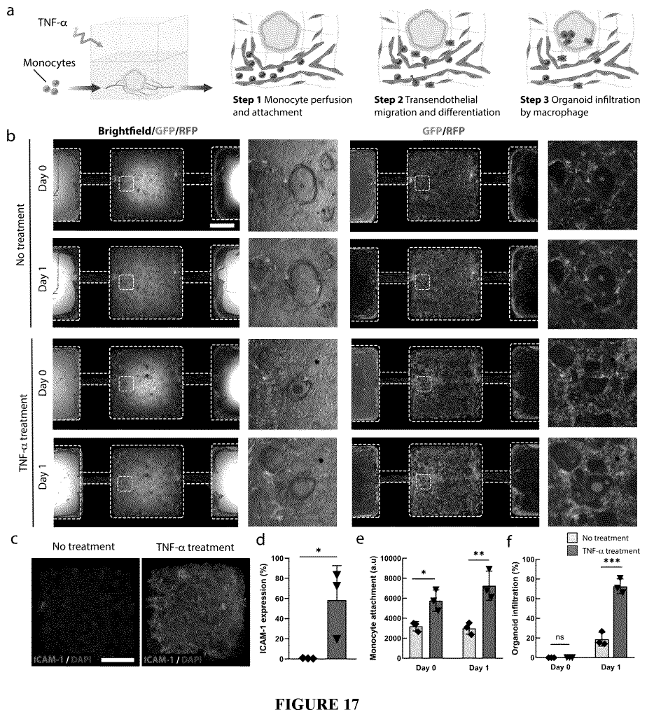

To demonstrate the importance of vasculature, a scenario of colon inflammation was assessed as a model example, which involves the complex interaction between circulating monocytes, endothelium, and colon epithelium. Release of TNF-α inflammatory cytokine during colon inflammation triggered the expression of ICAM-1 surface receptor on the vascular endothelium. ICAM-1 receptor then facilitated the attachment of circulating monocytes on the vascular endothelium. Adhered monocyte then went through transendothelial migration, followed by differentiation into macrophages and lastly, infiltration into the colon epithelium (FIG. 17a). This entire process was captured and visualized in the model. With static organoid culture, even though it is possible to embed monocytes in a matrix around the colon organoids, the concentration of monocytes will have to be controlled manually in an arbitrary way where the intravascular recruitment of monocytes which plays a crucial role in the amplification of inflammatory response is missing. In addition, without transendothelial migration, monocytes will have to be artificially activated to differentiate into macrophages with M-CSF, which is not physiological. It is well known that the endothelium niche plays a vital role in the activation and phenotypic transformation of macrophages.

It was demonstrated that the extent of monocyte recruitment is correlated to the extent of inflammation and TNF-α release by simply perfusing circulating monocytes through the vasculature emulating the exact process that happens in the body without the need for artificially activating the monocytes with M-CSF (FIG. 17b-e). With TNF-α stimulation, most perfused monocytes were quickly captured by the microvasculature, while most monocytes passed through the network in non-stimulated vasculature. After one day incubation, it was found that the monocyte-differentiated macrophages were able to infiltrate nearly 80% of the colon organoids with TNF-α stimulation (FIG. 170. Specifically, the macrophages were observed to have a strong tendency to aggregate around the cell debris produced by colon organoids, which correctly correspond with the scavenger function of macrophages. Given the critical role of the vasculature in the recruitment and activation of circulating monocytes and the amplification of inflammatory response, this is a biological process that cannot be accurately replicated with static organoid culture alone. Therefore, this platform may be a useful tool to expand the application of organoids to new biological processes that involve vasculature and the interplay between vasculature and epithelium.

### Discussion

Many organ-on-a-chip systems require dissociating organoids and then seeding the heterogeneous cell population into pre-defined biological structures (e.g., a membrane that mimics tissue interface) that physically constrict the growing tissue and restrict 3D biological remodeling that organoids offer. Therefore, there is a need to vascularize and perfuse organoids as is without cell dissociation or fragmentation to preserve the organ-level architecture and the remodeling capability of the organoids. The key challenge addressed here is the incorporation of perfusable vasculature that could guide the development of organoids without using physical structures to artificially define and restrict biological structure and remodeling. The microvasculature herein was found to be in close proximity to the organoids and physically intertwine with the organoids. Active flow circulation through the vasculature around the organoids may enhance mass transport across the organoid epithelium and improve organoid function. This cell culture device provides a scalable, robust, and cost-effective manufacturing method of fabricating perfusable vascular networks, and optionally vascularize spheroids and/or organoids within the network.

It is important to note that although the self-assembled vascular network described herein does not have uniform structures and flow rates, neither do native vessels. The level of heterogeneity observed herein makes the biological model more physiological and certainly more interesting to study. Furthermore, the vessel structures are not static and can also vary from day to day. The self-assembled vascular network is constantly making structural adjustments in response to flow and to nearby organoids, which is a valuable physiological feature that cannot be acquire if uniformity is forced on the system.

The platform also allows the organoids to be placed both inside and on top of the gel. However, in the example described herein, embedding the organoids inside the gel was chosen to introduce more contact areas between the organoids and the matrix to provide the vessels with more opportunities to intertwine with the organoids. In addition, supporting cells like fibroblasts were also incorporated in the gel. This embedding strategy is especially important to smaller epithelial organoids that do not have its own matrix. Without a supporting matrix, it will be difficult for the vasculature to grow upwards into the organoids if most parts of the organoids are exposed in suspension. For tissue explants or larger organoids, surface vascularization might be sufficient as the vessels will be able to grow into the organoids in the presence of significant tissue mass and matrix within the organoids. A potential advantage of surface vascularization is that the organoids will likely be less sensitive to the choice of the matrix, perhaps allowing the vascular environment and the organoids to be decoupled.

Organ-specific endothelial cells may also be used for the vascular assembly to provide a more organ-specific microenvironment around the organoids⁹. If it is critical to provide unidirectional recirculating flow in the biological model, the fluid circuit inside the plate can be adapted based on recently published strategy²² or the cap of the plate could be modified to include an array of microfluidic pumps to recirculate the media back to the inlets²³. The shear stress achieved in the microvasculature was still significantly below the physiological range. If shear stress is an important parameter to study, the height of the well plate could be increased to apply a higher pressure gradient. Furthermore, the incorporation of pericytes and smooth muscles has been shown to decrease vessel diameter, which will help increase shear stress inside the microvessels. Considering the large footprint of the 384-well plate, connecting multiple types of organoids between different wells through the perfusable vasculature is feasible even without compromising the experimental throughputs.

### Example 4. Compartmentalized Cell Culture System

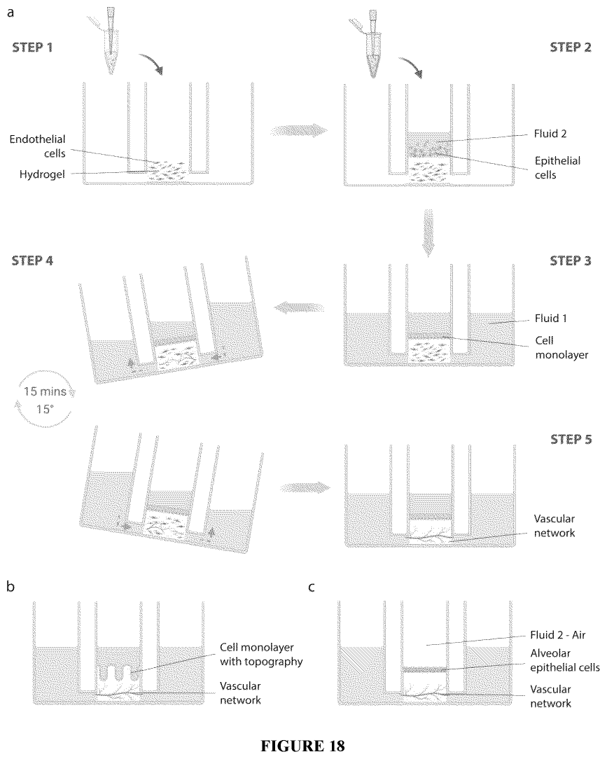

Although organoids contain sophisticated 3D tissue structures, the internal lumen of an organoid is not accessible, making it impossible to directly measure the epithelial barrier of an organoid. This issue can be overcome in this cell culture system by changing the way the organoid epithelial cells are seeded. This cell culture device can be compartmentalized in such a way to embed a perfusable vascular network inside the hydrogel matrix while having a monolayer of epithelial cells dissociated from organoids on the surface of the hydrogel (FIG. 18). This configuration effectively compartmentalizes the middle well from the inlet and outlet wells. By placing dextran dye in the middle well above the epithelial monolayer and measuring its concentration in the outlet and inlet wells over time, the permeability of the epithelial barrier can be quantified. In addition, it is possible to introduce topographies on the surface of the hydrogel during the gelation process using an insert with pre-patterned topographies. Furthermore, it is also possible to produce an air-liquid interface on the surface of the gel seeded with cells, such as lung airway cells, alveolar cells, or skin cells by removing all the media from the middle well. In this scenario, the cells at the liquid interface will be supported by culture media perfused inside the hydrogel matrix. A broad range of tissue models can be developed in this format, and epithelial barrier functions can be directly measured.

### Example 5. Topographical Guidance for Blood Vessel Assembly

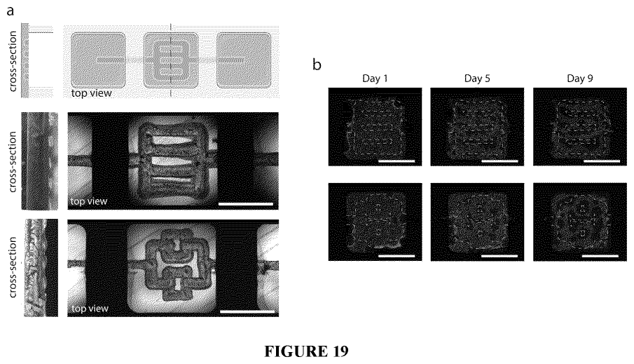

While the design of the cell culture device allows cells to form a network with random structures, it was found that a pattern of connected grooves at the bottom of the well (e.g. created using a standard milling technique) can guide the self-organization of the assembled vascular network (FIG. 19a). For example, it was demonstrated that grooves positioned in parallel results in aligned blood vessels while bifurcated grooves resulted in bifurcated vascular networks (FIG. 19b). This patterning strategy allows for additional control over the structure of the self-assembled blood vessels without the need for patterning cells and/or guiding cell assembly using a 3D printer. This strategy can also be used to support the guided assembly of non-endothelial cells to form elongated structures, such as liver cords, from hepatocytes, or branched structures, such as bile ducts, from cholangiocytes.

### Example 6. Biochemical Gradient Generation

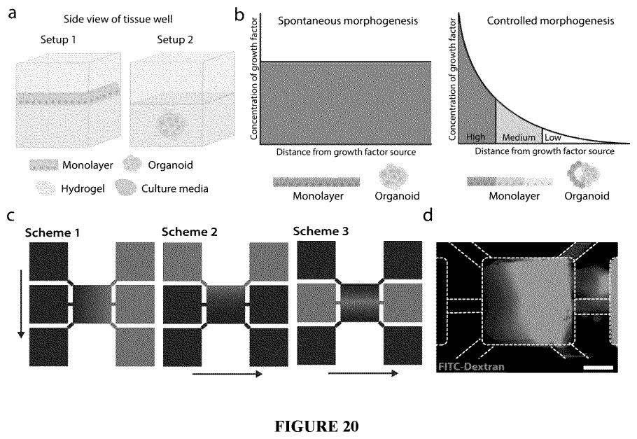

During organ development, cells encounter gradients of various growth factors that direct the fate of these cells to form complex organ structures. The local concentration of growth factors that each cell encounters can profoundly affect cell function and specialization. However, conventional cell culture systems do not allow for precise spatial control of these biochemical gradients that regulate the physiological developmental process. To enable this ability using the cell culture system described herein, multiple microchannels may be introduced to connect multiple adjacent wells to form a single perfusable unit (FIG. 20). For example, each unit can have three inlets and three outlets which can be perfused independently. This design allows the introduction of various biochemical gradients within the hydrogel in different patterns by simply adding the desired growth factor or biochemicals to specific inlets, outlets, and/or changing the direction of interstitial flow within the hydrogel. Cells or organoids either cultured on the surface of the gel or embedded inside the gel can be exposed to a specific biochemical gradient over time. The pattern of the biochemical gradient can also be easily changed during the experiment and culture period. In response to these gradients, cells will differentiate and give rise to specific tissue architectures. Building such sophisticated cellular microenvironments to accurately mimic organ morphogenesis in vitro could provide new insights into organ development and regeneration.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### CITATIONS

Zhang, B.; Korolj, A.; Lai, B. F. L.; Radisic, M., Advances in organ-on-a-chip engineering. Nature Reviews Materials 2018, 1 (3), 257-278.
Huh, D.; Matthews, B. D.; Mammoto, A.; Montoya-Zavala, M.; Hsin, H. Y.; Ingber, D. E., Reconstituting organ-level lung functions on a chip. Science 2010, 328 (5986), 1662-1668.
Huh, D.; Leslie, D. C.; Matthews, B. D.; Fraser, J. P.; Jurek, S.; Hamilton, G. A.; Thorneloe, K. S.; McAlexander, M. A.; Ingber, D. E., A human disease model of drug toxicity—induced pulmonary edema in a lung-on-a-chip microdevice. Science Translational Medicine 2012, 4 (159), 159ra147-159ra147.
Blundell, C.; Yi, Y. S.; Ma, L.; Tess, E. R.; Farrell, M. J.; Georgescu, A.; Aleksunes, L. M.; Huh, D., Placental Drug Transport-on-a-Chip: A Microengineered In Vitro Model of Transporter-Mediated Drug Efflux in the Human Placental Barrier. Advanced healthcare materials 2018, 7 (2), 1700786.
Kim, S.; Lee, H.; Chung, M.; Jeon, N. L., Engineering of functional, perfusable 3D microvascular networks on a chip. Lab on a Chip 2013.
Chung, M.; Kim, S.; Ahn, J.; Lee, S.; Jeon, N. L., Interstitial flow regulates angiogenic response and phenotype of endothelial cells in a 3D culture model. Lab on a Chip 2016, 16, 4189-4199.
Chung, M.; Lee, S.; Lee, B. J.; Son, K.; Jeon, N. L.; Kim, J. H., Wet-AMD on a Chip: Modeling Outer Blood-Retinal Barrier In Vitro. Advanced Healthcare Materials 2017.
Oh, S.; Ryu, H.; Tahk, D.; Ko, J.; Chung, Y.; Lee, H. K.; Lee, T. R.; Jeon, N. L., “Open-top” microfluidic device for in vitro three-dimensional capillary beds. Lab on a Chip 2017, 17 (20), 3405-3414.
Lin, D. S. Y.; Guo, F.; Zhang, B., Modeling organ-specific vasculature with organ-on-a-chip devices. Nanotechnology 2018, 3 (2).
Dutta, D., Heo, I. & Clevers, H. Disease modeling in stem cell-derived 3D organoid systems. Trends in molecular medicine 23, 393-410 (2017).
Clevers, H. Modeling development and disease with organoids. Cell 165, 1586-1597 (2016). Lancaster, M. A. & Knoblich, J. A. Organogenesis in a dish: modeling development and disease using organoid technologies. Science 345, 1247125 (2014).
Takebe, T., Zhang, B. & Radisic, M. Synergistic Engineering: Organoids Meet Organs-on-a-Chip. Cell Stem Cell 21, 297-300 (2017).
Wiedeman, M. P., Dimensions of blood vessels from distributing artery to collecting vein. Circulation Research 1963, 12 (4), 375-378.
Guzzardi, M. A.; Vozzi, F.; Ahluwalia, A. D., Study of the crosstalk between hepatocytes and endothelial cells using a novel multicompartmental bioreactor: a comparison between connected cultures and cocultures. Tissue Engineering Part A 2009, 15 (11), 3635-3644.
Zhang, B.; Radisic, M., Organ-level vascularization: The Mars Mission of Bioengineering. The Journal of Thoracic and Cardiovascular Surgery 2019.
Stevens, K. R.; Scull, M. A.; Ramanan, V.; Fortin, C. L.; Chaturvedi, R. R.; Knouse, K. A.; Xiao, J. W.; Fung, C.; Mirabella, T.; Chen, A. X., In situ expansion of engineered human liver tissue in a mouse model of chronic liver disease. Science translational medicine 2017, 9 (399), eaah5505.
McGuigan, A. P.; Sefton, M. V., Vascularized organoid engineered by modular assembly enables blood perfusion. Proceedings of the National Academy of Sciences 2006, 103 (31), 11461-11466.
Kim, B. S.; Kim, H.; Gao, G.; Jang, J.; Cho, D.-W., Decellularized extracellular matrix: a step towards the next generation source for bioink manufacturing. Biofabrication 2017, 9 (3), 034104.
Marcu, R.; Choi, Y. J.; Xue, J.; Fortin, C. L.; Wang, Y.; Nagao, R. J.; Xu, J.; MacDonald, J. W.; Bammler, T. K.; Murry, C. E., Human Organ-Specific Endothelial Cell Heterogeneity. iScience 2018, 4, 20-35.
Jang, K.-J.; Otieno, M. A.; Ronxhi, J.; Lim, H.-K.; Ewart, L.; Kodella, K. R.; Petropolis, D. B.; Kulkarni, G.; Rubins, J. E.; Conegliano, D., Reproducing human and cross-species drug toxicities using a Liver-Chip. Science translational medicine 2019, 11 (517).
Wang, Y. I.; Shuler, M. L., UniChip enables long-term recirculating unidirectional perfusion with gravity-driven flow for microphysiological systems. Lab on a Chip 2018.
Tan, K. et al. A high-throughput microfluidic microphysiological system (PREDICT-96) to recapitulate hepatocyte function in dynamic, re-circulating flow conditions. Lab on a Chip 19, 1556-1566 (2019).

1. An open chamber for cell culture, the chamber comprising an inlet and an outlet for non-tangential flow of fluid from the inlet to the outlet.

2. The open chamber of claim 1, wherein the chamber is an open cylinder.

3. The open chamber of claim 1 or 2, wherein the chamber comprises an open top and a closed bottom and wherein the open top and closed bottom have substantially equal diameters.

4. The open chamber of any one of claims 1 to 3, wherein the chamber does not comprise a sacrificial material.

5. The open chamber of any one of claims 1 to 4, wherein the chamber does not comprise a membrane.

6. The open chamber of any one of claims 1 to 5, wherein the inlet and/or outlet is a channel.

7. The open chamber of claim 6, wherein the channel is about 0.5 to about 3 times as wide as it is tall.

8. The open chamber of claim 7, wherein the channel is about 200 microns wide and about 100 to about 200 microns tall.

9. The open chamber of any one of claims 1 to 8, wherein the inlet leads to an inlet chamber and/or the outlet leads to an outlet chamber.

10. The open chamber of claim 9, wherein the inlet chamber and/or the outlet chamber are open or closed.

11. The open chamber of any one of claims 1 to 10, comprising at least two inlets and/or at least two outlets.

12. The open chamber of any one of claims 1 to 11, wherein the open chamber comprises a patterned base.

13. The open chamber of claim 12, wherein the patterned base comprises connected grooves for guiding self-assembly of cultured cells.

14. The open chamber of any one of claims 1 to 13, configured for unidirectional or bidirectional fluid flow from the inlet to the outlet.

15. The open chamber of any one of claims 1 to 14, further comprising a hydrogel on a bottom surface of the open chamber.

16. The open chamber of claim 15, wherein the hydrogel comprises a fibrin matrix, fibrin, Matrigel, collagen I, a decellularized matrix, or a combination thereof.

17. The open chamber of any one of claims 1 to 16, further comprising cells seeded into the open chamber.

18. An array comprising the open chamber of any one of claims 1 to 17 and at least one inlet chamber and at least one outlet chamber.

19. A multi-well plate comprising the array of claim 18.

20. The multi-well plate of claim 13, comprising a plurality of the arrays of claim 9.

21. A method for making a perfusable network of self-assembled cells, the method comprising applying a hydrogel and cells to the open chamber of any one of claims 1 to 17 and culturing the cells.

22. The method of claim 21, wherein the hydrogel and the cells are applied together as a mixture.

23. The method of claim 21, wherein the hydrogel and the cells are applied sequentially.

24. The method of any one of claims 21 to 23, further comprising flowing fluid from the inlet to the outlet.

25. A heterogenous perfusable network of self-assembled cells.

26. A cell culture system for constructing a perfusable network of self-assembled cells comprising a multi-well plate embedded with microchannels connecting a central well with at least one inlet well and at least one outlet well, the central well for culturing seeded cells within an extracellular matrix, wherein the perfusable network allows perfusion through the microchannels connecting the central well with at least one inlet well and at least one outlet well.

27. The system of claim 26, wherein the perfusable network is accessible from the top of the central well.

28. The system of claim 26 or 27, wherein the perfusable network is extractable from the top of the central well.

29. The system of any one of claims 26 to 28, wherein the multi-well plate comprises 6 wells, 12 wells, 24 wells, 96 wells, 384 wells, or 1536 wells.

30. The system of any one of claims 26 to 29, wherein the multi-well plate comprises of 2, 4, 8, 32, 128, or 512 central wells.

31. The system of any one of claims 26 to 30, wherein the perfusable network mimics a blood or lymph vessel network, the architecture of an organ or a tissue, or a cavity of an organ or a tissue.

32. The system of any one of claims 26 to 31, wherein the seeded cells comprise hepatocytes, pancreatic Islet cells, fibroblasts, chondrocytes, osteoblasts, endothelial cells, exocrine cells, smooth or skeletal muscle cells, myocytes, adipocytes, ectodermal cells, ductile cells, kidney cells, intestinal cells, parathyroid cells, thyroid cells, nerve cells, ocular cells, integumentary cells, pluripotent cells, stem cells, or combinations thereof.

33. The system of any one of claims 26 to 32, wherein the extracellular matrix is a hydrogel.

34. The system of any one of claims 26 to 33, wherein the extracellular matrix comprises collagen, fibrin, fibrinogen, basement membrane proteins, gelatinous protein mixture secreted by Engelbreth-Holm-Swarm (EHS) mouse sarcoma cells or a combination thereof.

35. The system of any one of claims 26 to 34, further comprising spheroids, organoids, or a combination thereof embedded within the perfusable network.

36. The system of any one of claims 26 to 35, wherein the perfusable network is for use in vitro for research and development.

37. The system of any one of claims 26 to 36, wherein the perfusable network is for use in vivo for cell therapy.

38. A method for constructing a perfusable network of self-assembled cells, the method comprising:
combining a plurality of cells and a gel matrix into a mixture,
transferring the mixture into the central well of the multi-well plate of any one of claims 26 to 37, and
allowing the plurality of cells to self-assemble into a perfusable network.

39. The method of claim 38, further comprising transferring spheroids, organoids, or a combination thereof into the central well with the mixture in step b).

40. The method of claim 39, further comprising removing the perfusable network from the central culture well of the cell culture device.

41. A perfusable network of self-assembled cells constructed using the method of claims 38 to 40, wherein the network mimics a blood or lymph vessel network, the architecture of an organ or a tissue, or a cavity of an organ or a tissue.

42. The perfusable network of claim 41, further comprising spheroids, organoids, or a combination thereof embedded within the network.

43. The perfusable network of claim 41 or 42, wherein the network is for use in vitro for research and development.

44. The perfusable network of any one of claims 41 to 43, wherein the network is for use in vivo for cell therapy.
